SEQUENCE LISTING (13 sequences)

SEQ ID NO: 1 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 2 - protein, 996 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNVRLARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 3 - DNA, 20 bp, Artificial Sequence
  source: /note=“Description of Artificial Sequence Synthetic primer”
caaattcctccctgttgctg

SEQ ID NO: 4 - DNA, 19 bp, Artificial Sequence
  source: /note=“Description of Artificial Sequence Synthetic primer”
ggtgggagatgttggtctg

SEQ ID NO: 5 - DNA, 21 bp, Artificial Sequence
  source: /note=“Description of Artificial Sequence Synthetic primer”
gctggtcgtcgacaacggctc

SEQ ID NO: 6 - DNA, 25 bp, Artificial Sequence
  source: /note=“Description of Artificial Sequence Synthetic primer”
caaacatgatctgggtcatcttttc

SEQ ID NO: 7 - protein, 6 aa, Artificial Sequence
  source: /note=“Description of Artificial Sequence Synthetic peptide”
RTDFRE

SEQ ID NO: 8 - DNA, 20 bp, Artificial Sequence
  source: /note=“Description of Artificial Sequence Synthetic primer”
ctctcgggaactcccactta

SEQ ID NO: 9 - DNA, 20 bp, Artificial Sequence
  source: /note=“Description of Artificial Sequence Synthetic primer”
tgcagatgatccaggtgact

SEQ ID NO: 10 - DNA, 20 bp, Artificial Sequence
  source: /note=“Description of Artificial Sequence Synthetic primer”
gcccaaatgtgcgtatacct

SEQ ID NO: 11 - DNA, 20 bp, Artificial Sequence
  source: /note=“Description of Artificial Sequence Synthetic primer”
cccagcacagtatgggaact

SEQ ID NO: 12 - DNA, 20 bp, Artificial Sequence
  source: /note=“Description of Artificial Sequence Synthetic primer”
acacttcgaagctggaaagc

SEQ ID NO: 13 - DNA, 20 bp, Artificial Sequence
  source: /note=“Description of Artificial Sequence Synthetic primer”
ggggaacttcctgactaggg

(19) United States
(12) Patent Application Publication
(10) Pub. No.: US 2013/0156764 A1
(43) Pub. Date: Jun. 20, 2013

(54) NEUTRALIZATION OF FLT3 LIGAND AS A LEUKEMIA THERAPY

(71) Applicants: Mark J. Levis, Phoenix, MD (US); Donald Small, Pikesville, MD (US)

(72) Inventors: Mark J. Levis, Phoenix, MD (US); Donald Small, Pikesville, MD (US)

(73) Assignee: The Johns Hopkins University, Baltimore, MD (US)

(21) Appl. No.: 13/634,439

(22) PCT Filed: Mar. 14, 2011
(86) PCT No.: PCT/US2011/028315

§ 371 Date: Feb. 25, 2013

Related U.S. Application Data
(60) Provisional application No. 61/313,245, filed on Mar. 12, 2010.
(60) Provisional application No. 61/426,604, filed on Dec. 23, 2010.

(51) Int. Cl. A61K 39/395; A61K 31/553; A61K 31/44; A61K 31/496; A61K 31/7068; A61K 31/517; A61K 31/404; A61K 31/7048; A61K 31/704; A61K 31/136; A61K 38/45; A61K 31/5377

(52) U.S. Cl. CPC A61K 39/39558; A61K 39/3955; A61K 38/45; A61K 31/553; A61K 31/44; A61K 31/496; A61K 31/5377; A61K 31/517; A61K 31/404; A61K 31/7048; A61K 31/704; A61K 31/136; A61K 31/7068
USPC 424/134.1; 424/158.1; 514/19.6; 424/138.1

(57) ABSTRACT

Disclosed herein are methods and compositions for treating leukemia and preventing leukemia relapse related to the administration of agents that inhibit the binding of FLT3 ligand to FLT3.

### RELATED APPLICATIONS

This application claims the benefit of priority under 35 U.S.C. §119(e) to Provisional Application Ser. No. 61/313,245, filed Mar. 12, 2010 and Provisional Application Ser. No. 61/426,604, filed Dec. 23, 2010, the contents of which are all incorporated by reference in their entirety.

### BACKGROUND OF THE INVENTION

FLT3 is a member of the class III receptor tyrosine kinase family that is normally expressed by CD34⁺ hematopoietic stem/progenitor cells, dendritic cells, brain, placenta and gonads. FLT3 is also frequently expressed on the cancer cells of patients with leukemia, including in most cases of acute myeloid leukemia (AML), B-precursor acute lymphoblastic leukemia (ALL) and blast crisis chronic myelocytic leukemia (CML).

Mutations to the FLT3 receptor that result in ligand-independent activation are frequently present in patients with certain forms of leukemia. For example, internal tandem duplication (ITD) mutations are present in approximately 24% of patients with AML, while mutations in the activation loop of the tyrosine kinase domain (TKD) occur in about 8% of patients with AML. Both types of mutations lead to constitutive activation of FLT3 in a ligand-independent manner, which results in the transformation of hematopoietic cell lines.

Leukemia patients who harbor activating FLT3 mutations have an exceptionally poor prognosis. Over the past decade, efforts have been underway to develop FLT3 kinase inhibitors in hopes of improving outcomes of these patients. Several agents have been studied both as a monotherapy and in combination with chemotherapy, but the results have only been modestly successful. There is therefore great need for new compositions and methods for the treatment of leukemia and the prevention of relapse in treated patients.

### SUMMARY

Disclosed herein are novel compositions and methods for the treatment of leukemia, including leukemia harboring activated FLT3 mutations, and the prevention of leukemia relapse following FLT3 kinase inhibitor and/or chemotherapy treatment.

In certain embodiments, the method of treating leukemia includes the administration of a therapeutically effective amount of an agent that inhibits the tyrosine kinase activity of FLT3 (e.g., a small molecule such as lestaurtinib, midostaurin, sorafenib, KW-2449 and/or AC220) and the administration of a therapeutically effective amount of an agent that inhibits the binding of FLT3L to FLT3 (e.g., an FL antibody, a FLT3 antibody, and/or a FL binding polypeptide). In some embodiments, the subject is also administered chemotherapy.

In some embodiments, the method of preventing a leukemia relapse in a subject who had previously been treated for leukemia with an agent that inhibits the tyrosine kinase activity of FLT3 (e.g., a small molecule such as lestaurtinib, midostaurin, sorafenib, KW-2449 and/or AC220) and/or chemotherapy includes the administration of a therapeutically effective amount of an agent that inhibits the binding of FLT3L to FLT3 (e.g., an FL antibody, a FLT3 antibody, and/or a FL binding polypeptide).

In certain embodiments, the instant invention relates to a kit for treating leukemia that includes an agent that inhibits the tyrosine kinase activity of FLT3 (e.g., a small molecule such as lestaurtinib, midostaurin, sorafenib, KW-2449 and/or AC220) and an agent that inhibits the binding of FLT3L to FLT3 (e.g., an FL antibody, a FLT3 antibody, and/or a FL binding polypeptide).

### BRIEF DESCRIPTION OF THE FIGURES

FIG. 1 shows the human FL amino acid sequence (SEQ ID NO: 1).

FIG. 2 shows the human FLT3 amino acid sequence (SEQ ID NO: 2).

FIG. 3 shows a table depicting a comparison of lestaurtinib trough plasma levels and FLT3 inhibitory activity from Day 15 samples of AML15 and Cephalon 204 trial patients. All patients received 80 mg twice daily lestaurtinib, started after completion of chemotherapy (which began on Day 1). Mean FLT3 activity represents the mean of values obtained from the PIA assay for these time points. Percent FLT3 inhibited refers to the percentage of patients for whom FLT3 was inhibited to less than the target of 15% of baseline or less.

FIG. 4 shows the plasma FLT3 ligand (FL) levels from clinical trial patients. (A) Plasma samples obtained on Day 15 of following induction of therapy for newly diagnosed (left) versus relapsed (right) FLT3 mutant AML patients were assayed for FL by ELISA. (B) FL levels from plasma samples obtained from newly diagnosed FLT3 mutant AML patients. Course 1 was induction, courses 2-4 were consolidation. The course 1 samples correspond to the samples on the left in (A). (C) FL plasma levels from individual patients in the Cephalon 204 trial at 3 different time points during therapy.

FIG. 5 shows that FL impairs inhibition of FLT3 autophosphorylation in vitro. (A) Molm14 cells were exposed to increasing concentrations of lestaurtinib in the presence of 0, 1, and 3 ng/mL of FL in plasma for 2 hours. FLT3 autophosphorylation was then evaluated by immunoblotting. The bands were analyzed by densitometry and plotted. (B) Molm14 cells were incubated in plasma with 10 μM of the indicated drug and 0 (“−”) or 3 (“+”) ng/mL of FL for 2 hours. FLT3 autophosphorylation was then evaluated by immunoblotting.

FIG. 6 shows that FL impairs the cytotoxic effects of FLT3 inhibitors. Molm14 cells were incubated with increasing concentrations of the indicated drugs for 48 hours in the presence of 0, 1, 3, and 10 ng/mL of FL. Cell viability was then determined using an MTT assay. Results are plotted as percent DMSO control.

FIG. 7 shows a table depicting the cytotoxicity assay results for FLT3 inhibitors in the presence or absence of FL. Molm14 cells were incubated for 48 hours with increasing concentrations of FLT3 inhibitors in the presence increasing concentrations of FL, and then assayed for cytotoxic effect using an MTT assay. The concentration of drug for which the optical density was reduced to 50% or 80% of its baseline was determined using linear regression analysis of the dose response curves after linear transformation using an exponential model.

FIG. 8 shows that FL impairs the cytotoxic effects of FLT3 inhibitors in primary AML samples. Three different primary AML blast samples, each harboring a FLT3/ITD mutation, were incubated with increasing concentrations of the indicated drugs for 48 hours in the presence of FL as in FIG. 6. Cell viability was then determined using the MTT assay. Results are plotted as percent DMSO control.

FIG. 9 shows that FL impairs inhibition of FLT3 autophosphorylation in vivo. (A) Plasma samples from two individual patients treated in the AML15 trial were collected at different time points and then assessed for FLT3 inhibitory activity (PIA assay). Cells were exposed to plasma for 3 hours, and then lysed. FLT3 was immunoprecipitated, subject to electrophoresis, and transferred to a membrane. The blot was probed with antiphosphotyrosine (upper row), then stripped and re-probed with anti-FLT3 (lower row). FL and lestaurtinib levels were determined from the same plasma samples. (B) Plasma was collected from a single, newly diagnosed AML patient at different time points following diagnosis and treatment with induction chemotherapy (cytarabine, daunorubicin, and etoposide). The plasma was assayed for FL levels by ELISA and plotted (open circles). In parallel, AC220 was added to a concentration of 2 μM for each time point, and used to incubate Molm14 cells for 2 hours. Each sample was then assayed for FLT3 inhibitory activity as in (A). The densitometric analysis of the phospho-FLT3 blot (upper blot) was plotted in solid circles on the graph.

FIG. 10 shows that FL addition leads to increased phosphorylation of FLT3 in FL^{-/-} MEF cells expressing FLT3/ITD or TKD mutants. (A) Total cellular RNA was extracted from cell lines. RNA (50-100 ng) was reverse transcribed and amplified using primer pairs for murine FL and actin. PCR products were resolved on 1% agarose gel in the presence of ethidium bromide, and photographs were taken under UV transillumination. (B) Cells were cultured in serum-free medium for 16 hours, washed once with PBS and stimulated with FL (100 ng/mL) for 15 minutes. Total cellular protein extracts derived from 1×10⁷ cells were immunoprecipitated with anti-FLT3 antibody. The immunoprecipitates were resolved by 8% SDS-PAGE and subjected to immunoblot analysis with the antiphosphotyrosine antibody 4G10 (top panel). The same blot was stripped and reprobed with anti-FLT3 antibody (bottom panel).

FIG. 11 shows that stable FL expression results in enhanced FLT3 phosphorylation in FL^{-/-} MEF cells expressing FLT3/ITD and FLT3/TKD mutants. (A) FL^{-/-} MEF cells expressing wt and mutant forms of FLT3 with and without stable expression of FL were washed with PBS, plated at 5×10⁵ cells/mL, and cultured for 24 hours in serum-free medium. Cell culture supernatant was then collected by centrifugation and FL concentration was assessed by ELISA assay. (B) Cells were cultured in fresh serum free medium for 16 hours and washed with PBS once before stimulation with FL (100 ng/mL for 15 minutes). Total cellular protein extracts derived from 1×10⁷ cells were immunoprecipitated with anti-FLT3 antibody. The immunoprecipitates were resolved by 8% SDS-PAGE and subjected to immunoblot analysis with the antiphosphotyrosine antibody 4G10 (top panel). The same blot was stripped and reprobed with anti-FLT3 antibody (bottom panel).

FIG. 12 shows that addition of FL results in an increased phosphorylation of FLT3 and resistance to apoptosis in TF1 cells expressing FLT3/ITD and FLT3/TKD mutations. (A) Cells were cultured in serum-free medium for 16 hours and washed once with PBS before stimulation with FL (100 ng/mL for 15 minutes). Total cellular protein extracts derived from 1×10⁷ cells were immunoprecipitated with anti-FLT3 antibody. The immunoprecipitates were resolved by 8% SDS-PAGE and subjected to immunoblot analysis with the antiphosphotyrosine antibody 4G10. The same blot was stripped and reprobed with anti-FLT3 antibody. Total protein lysates (50 μg) were resolved by 10% SDS-PAGE and subjected to immunoblot analysis with anti-phosphoSTAT5, anti-phospho-AKT, and anti-phospho-MAPK antibody. The same blot was stripped and reprobed with anti-STAT5, anti-AKT, anti-MAPK, and anti-HSP90 antibodies. (B) Cells were cultured in serum-free medium for 48 hours in the presence and absence of FL (100 ng/mL). The cells were then stained with Annexin V and 7AAD followed by FACS analysis. The result shown here is representative of three independent experiments. (C) Cells were cultured in serum free medium for 4 days in the presence and absence of FL (100 ng/mL). Viable cell numbers were assessed by trypan blue exclusion assay. The result shown here is representative of three independent experiments (Bars represent standard deviation in the figure).

FIG. 13 shows that exogenous FL augments FLT3 phosphorylation and provides a survival advantage to BaF3 cells expressing FLT3 mutants. (A) Cells were cultured in serum-free medium for 16 hours and washed once with PBS once before stimulation with FL (100 ng/mL for 15 minutes). Total cellular protein extracts derived from 1×10⁷ cells were immunoprecipitated with anti-FLT3 antibody. The immunoprecipitates were resolved by 8% SDS-PAGE and subjected to immunoblot analysis with antiphosphotyrosine antibody 4G10. The same blot was stripped and reprobed with anti-FLT3 antibody. Total protein lysates (50 μg) were resolved by 10% SDS-PAGE and subjected to immunoblot analysis with anti-phosphoSTAT5, anti-phospho-AKT, and anti-phospho-MAPK antibody. The same blot was stripped and reprobed with anti-STAT5, anti-AKT, anti-MAPK, and anti-HSP90 antibodies. (B) Cells were cultured in serum free medium for 4 days in the presence and absence of FL (100 ng/mL). Survival was assessed by MTT assay. The result shown here is representative of three independent experiments (Bars represent standard deviation in the figure). (C) Cells were cultured in serum free medium for 4 days in the presence and absence of FL (100 ng/mL). Viable cell numbers were assessed by trypan blue exclusion assay. The result shown here is representative of three independent experiments (Bars represent standard deviation in the figure).

FIG. 14 shows that addition of FL leads to full activation of FLT3 receptors and increased survival of MV411 cells. (A) Cells were cultured in serum-free medium for 16 hours and washed once with PBS once before stimulation with FL (100 ng/mL for 15 minutes). Total cellular protein extracts derived from 1×10⁷ cells were immunoprecipitated with anti-FLT3 antibody. The immunoprecipitates were resolved by 8% SDS-PAGE and subjected to immunoblot analysis with antiphosphotyrosine antibody 4G10. The same blot was stripped and reprobed with anti-FLT3 antibody. Total protein lysates (50 μg) were resolved by 10% SDS-PAGE and subjected to immunoblot analysis with anti-phosphoSTAT5, anti-phospho-AKT, and anti-phospho-MAPK antibody. The same blot was stripped and reprobed with anti-STAT5, anti-AKT, anti-MAPK, and anti-HSP90 antibodies. (B) Cells were cultured in serum free medium for 4 days in the presence and absence of FL (100 ng/mL). The survival of the cells was assessed by MTT assay. The result shown here is representative of three independent experiments (Bars represent standard deviation in the figure).

FIG. 15 shows that FL addition results in increased phosphorylation of FLT3 receptors and prolongs survival of primary AML blasts expressing homozygous FLT3/ITD mutations. (A) Frozen AML blasts were thawed and stimulated with 100 ng/mL FL for 15 minutes in serum-free medium. Total cellular protein extracts were immunoprecipitated with anti-FLT3 antibody. The immunoprecipitates were resolved by 8% SDS-PAGE and subjected to immunoblot analysis with the antiphosphotyrosine antibody 4G10. The same blot was stripped and reprobed with anti-FLT3 antibody. Total protein lysates (50 μg) were resolved by 10% SDS-PAGE and subjected to immunoblot analysis with anti-phosphoSTAT5, anti-phospho-AKT, and anti-phospho-MAPK antibodies. The same blot was stripped and reprobed with anti-STAT5, anti-AKT, anti-MAPK, and anti-HSP90 antibodies. (B) Frozen AML blasts were thawed and cultured in complete medium for 16 hours, separated by Ficoll-Hypague density gradient centrifugation, and incubated in fresh medium containing 10% FBS with and without FL (100 ng/mL) for 4 days. Survival of the cells was assessed by MTT assay. The result shown here is representative of two independent experiments (Bars represent standard deviation in the figure).

FIG. 16 shows that exogenous FL increases the clonogenic potential of FL^{-/-} primary hematopoietic stem/progenitor cells expressing FLT3/ITD and FLT3/TKD mutations. Bone marrow was harvested from FL^{-/-} mice, lineage depleted, and transduced with EF1-ITD-UBC-GFP, EF1-TKD-UBC-GFP, or EF1-UBC-GFP control virus by spin-infection. GFP positive cells were then sorted by FACS. 10³ cells were plated into triplicate 35 mm Petri dishes in 1 ml of Methocult M3434. Colonies consisting of >35 cells were scored after 9 days. For colony-replating all cells from the previous culture were collected and replated at 1×10⁴ cells/plate in fresh methylcellulose medium. Cultures were replated three times and designated as 2^{nd} plating, 3^{rd} plating and 4^{th} plating in the figure. The result shown here is representative of two independent experiments (Bars represent standard deviation in the figure).

1. FIG. 17 shows that endogenous FL shortens the survival of FL^{+/+}ITD^{+/+} mice in comparison with FL^{-/-}ITD^{+/+} mice. Kaplan-Meier plot of survival of FL^{+/+}ITD^{+/+} mice (n=51) and FL^{-/-}ITD^{+/+} mice (n=32).

FIG. 18 shows a table depicting the disease phenotype of FL^{+/+}ITD^{+/+} and FL^{-/-} ITD^{+/+} mice.

### DETAILED DESCRIPTION

### General

Disclosed herein are novel compositions and methods for the treatment of leukemia and the prevention of leukemia relapse following FLT3 kinase inhibitor and/or chemotherapy treatment.

Leukemia patients often harbor FLT3 mutations, such as ITD mutations or TKD mutations, which result in a constitutively activated FLT3 receptor, even in the absence of ligand. Notably, leukemias expressing activating FLT3 mutations are among the most difficult to treat, and patients inflicted by such leukemias have an exceptionally poor prognosis. A number of small molecule FLT3 kinase inhibitors, including lestaurtinib, midostaurin, sorafenib, KW-2449 and AC220, have been tested for the treatment of FLT3 mediated leukemias with modest success. However, the use of such compounds is usually insufficient to achieve total remission. Thus, in an attempt to improve therapeutic outcomes, recent studies have focused on the combination of such FLT3 kinase inhibitors with conventional chemotherapy.

However, as described herein, chemotherapeutic treatment of leukemia patients results in increased levels of FLT3 ligand (FLT3L or FL) in the treated patient. Such elevated FL levels are able to increase the phosphorylation and activation of both wild-type and mutant FLT3 receptors, thereby increasing leukemia cell survival and/or decreasing leukemia cell apoptosis. Furthermore, FL at concentrations similar to those observed in chemotherapy patients inhibited the ability of small molecule FLT3 kinase inhibitors to prevent FLT3 autophosphorylation and induce cytotoxicity. Agents that are able to inhibit the binding of FL to FLT3, such as antibodies specific for FL or FLT3, are therefore useful, for example, for increasing the efficacy of small molecule FLT3 kinase inhibitors in the treatment of leukemia and the prevention of relapse in leukemia patients undergoing chemotherapy.

Thus, in certain embodiments, the instant invention relates to a method of treating a leukemia in a subject (e.g., AML, CML or ALL) that includes administering to the subject a therapeutically effective amount of a first agent that inhibits the tyrosine kinase activity of FLT3 (e.g., a small molecule such as lestaurtinib, midostaurin, sorafenib, KW-2449 and/or AC220) and administering to the subject a therapeutically effective amount of a second agent that inhibits the binding of FLT3L to FLT3 (e.g., an FL antibody, a FLT3 antibody, and/or a FL binding polypeptide). In some embodiments, the patient is also administered chemotherapy.

In some embodiments, the instant invention relates to a method of preventing a leukemia relapse in a subject (e.g., an AML, CML or ALL relapse) who had previously been treated for leukemia with a first agent that inhibits the tyrosine kinase activity of FLT3 (e.g., a small molecule such as lestaurtinib, midostaurin, sorafenib, KW-2449 and/or AC220) and/or chemotherapy, where the method includes the administration of a therapeutically effective amount of a second agent that inhibits the binding of FLT3L to FLT3 (e.g., an FL antibody, a FLT3 antibody, and/or a FL binding polypeptide).

In certain embodiments, the instant invention relates to a kit for treating leukemia in a subject (e.g., AML, CML or ALL) which includes a therapeutically effective amount of a first agent that inhibits the tyrosine kinase activity of FLT3 (e.g., a small molecule such as lestaurtinib, midostaurin, sorafenib, KW-2449 and/or AC220) and a therapeutically effective amount of a second agent that inhibits the binding of FLT3L to FLT3 (e.g., an FL antibody, a FLT3 antibody, and/or a FL binding polypeptide). In some embodiments the kit also includes a chemotherapy agent.

### DEFINITIONS

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent, such as an agent that inhibits FLT3 kinase activity, an agent that inhibits the binding of FLT3 to FL and/or a chemotherapeutic agent, to a subject, and includes, but is not limited to, administering by a medical professional and self-administering.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments. An “isolated antibody,” as used herein, refers to an antibody which is substantially free of other antibodies having different antigenic specificities. An isolated antibody may, however, have some cross-reactivity to other, related antigens.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

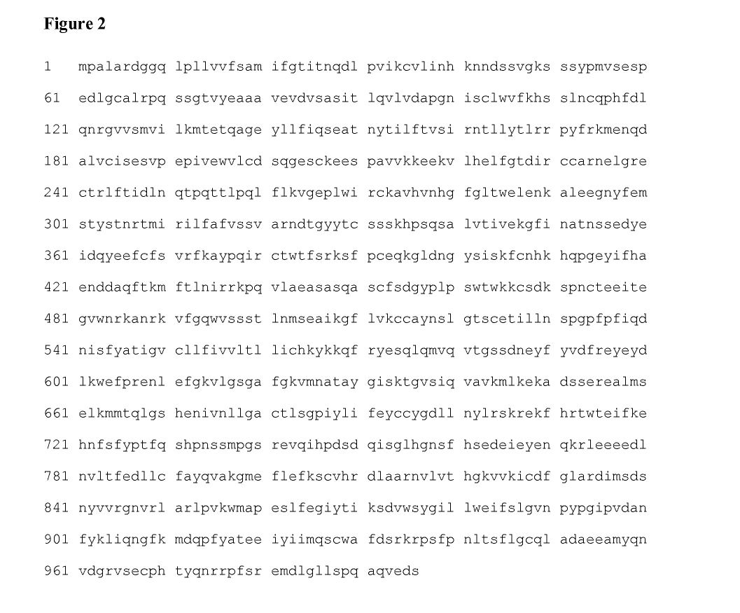

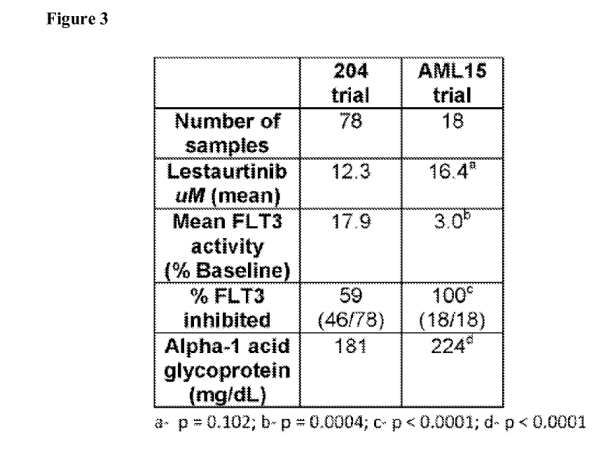

As used herein, the terms “FLT3” and “FLT3 receptor” are used interchangeably and refer to the fins-related tyrosine kinase, a member of the class III receptor tyrosine kinase family. FLT3 has also been known as FLK2, STK1 and CD135. The FLT3 receptor has of an extracellular region composed of five immunoglobulin-like (Ig-like) domains, has one transmembrane region, and has a cytoplasmic kinase domain split into two parts by a kinase-insert domain. The wild-type receptor is activated by the binding of FL to the extracellular domain, which induces homodimer formation in the plasma membrane and leads to autophosphorylation of the receptor. The activated receptor kinase subsequently phosphorylates and activates multiple cytoplasmic effector molecules in pathways involved in, for example, cellular apoptosis, proliferation, and differentiation. The FLT3 amino acid sequence is provided, for example, at GI:55959052 (human); GI:122937353 (mouse); and GI:281371429 (rat), each of which is incorporated by reference. The FLT3 nucleic acid sequence is provided, for example, at GI:121114303 (human); GI:122937352 (mouse); and GI:281371428 (rat), each of which is incorporated by reference. The human FLT3 amino acid sequence is also provided in FIG. 2. As used herein, the extracellular domain of FLT3 consists of about amino acids 24-541 of the FLT3 amino acid sequence. Ig-like domain 1 consists of about amino acids 80-183 of the FLT3 amino acid sequence. Ig-like domain 2 consists of about amino acids 184-271 of the FLT3 amino acid sequence. Ig-like domain 3 consists of about amino acids 272-370 of the FLT3 amino acid sequence. Ig-like domain 4 consists of about amino acids 371-451 of the FLT3 amino acid sequence. Ig-like domain 5 consists of about amino acids 452-541 of the FLT3 amino acid sequence.

As used herein, the term “FLT3 activating mutation” refers to mutations in the FLT3 protein that result in the constitutive and ligand-independent activation of the FLT3 receptor, such as ITD mutations and TKD mutations. Such mutations frequently occur in several forms of leukemia, including AML, ALL and MLL. Examples of activating FLT3 mutations are known in the art and can be found, for example, in Nakao et al., Leukemia 10:1911-1918 (1996); Yamamoto et al., Blood 97:2434-2439 (2001); and Bacher et al., Blood 111:2527-2537 (2008), each of which is hereby incorporated by reference in its entirety. As used herein, the terms “active mutant FLT3” and “activated mutant FLT3” refer to FLT3 proteins that have FLT3 activating mutations.

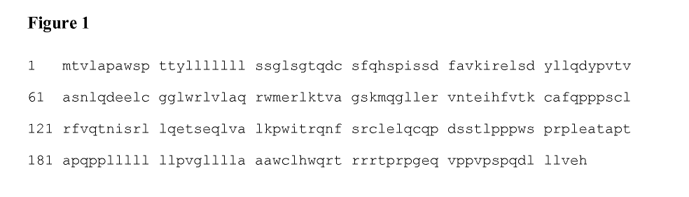

As used herein, the terms “FLT3 ligand,” “FLT3L” and “FL” are used interchangeably and refer to the fms-related tyrosine kinase ligand. The FLT3 ligand amino acid sequence is provided, for example, at GI:325197197 (human) and GI:227430331 (mouse), each of which is incorporated by reference. The human FLT3 ligand amino acid sequence is also provided in FIG. 1. The FLT3 ligand nucleic acid sequence is provided, for example at GI:325197196 (human) and GI:227430330 (mouse), each of which is incorporated by reference.

As used herein, the terms “FLT3 kinase inhibitor” and “inhibitor of FLT3 kinase activity” refers to an agent that inhibits the autophosphorylation activity of FLT3. Examples of FLT3 kinase inhibitors include, but are not limited to, lestaurtinib, midostaurin, sorafenib, KW-2449 and AC220. Other FLT3 kinase inhibitors are provided herein below. FLT3 kinase inhibitors may selectively inhibit FLT3 kinase activity, or they may inhibit the kinase activity of other proteins, such as other receptor tyrosine kinases. In general, FLT3 kinase inhibitors are able to inhibit the kinase activity of both wild-type FLT3 and activated mutant FLT3. The term “FLT3 kinase inhibitor” does not encompass FLT3 inhibitors that function by preventing FL from binding to FLT3.

As used herein, the term “FLT3 ligand inhibitor” refers to an agent that inhibits the ability of FL to bind to and activate FLT3. Examples of FLT3 ligand inhibitors include antibodies or antibody fragments that bind to FL, antibodies or antibody fragments that bind to FLT3, soluble polypeptides that bind to FL and small molecules that interfere with the binding of FL to FLT3.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body

“Pharmaceutically-acceptable salts” refers to the relatively non-toxic, inorganic and organic salts of compounds.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁸ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to administering the subject to a pharmaceutical treatment, e.g., the administration of a drug, such as a FLT3 kinase inhibitor and/or a FLT3 ligand inhibitor, such that at least one symptom of the disease is decreased or prevented from worsening.

### FLT3 Kinase Inhibitors

In certain embodiments the invention relates to the use of agents that inhibit the autophosphorylation and activation of FLT3 (FLT3 kinase inhibitors). In general, any FLT3 kinase inhibitor can be used in the methods disclosed herein. In some embodiments, the FLT3 kinase inhibitor is a small molecule. In some embodiments the FLT3 kinase inhibitor has an FLT3 IC₅₀ of no greater than 1 μM, 500 nM, 300 nM, 250 nM, 200 nM, 150 nM, 100 nM, 75 nM, 50 nM, 40 nM, 30 nM, 25 nM, 20 nM, 15 nM or 10 nM.

A number of FLT3 kinase inhibitors are known in the art. For example, in some embodiments, the FLT3 kinase inhibitor is selected from a group consisting of AG1295, AG1296, AGL2043, D64406, SU5416, SU5614, sunitnib (SU11248), MLN518, lestaurtinib (CEP-701), midostaurin (PKC412), Sorafenib, GTP-14564, Ki23819, CHIR-258, AC220 and KW-2449. In certain embodiments, the FLT3 kinase inhibitor is lestaurtinib, midostaurin, sorafenib, KW-2449 or AC220.

Lestaurtinib (CEP-701) is related to staurosporine and has a FLT3 IC₅₀ of about 2 nM. Lestaurtinib is described, for example, in Levis et al., Blood 99:3885-3891 (2002) and Smith et al., Blood 103:3669-3676 (2004), each of which is incorporated by reference in its entirety. Lestaurtinib has the following chemical structure:

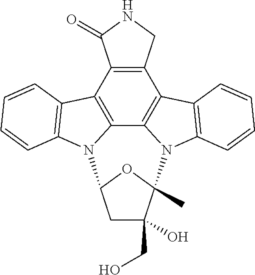 [SMILES: C[C@]12O[C@H](C[C@]1(O)CO)n1c3ccccc3c3c4c(c5c6ccccc6n2c5c31)CNC4=O]

Midostaurin (PKC412), like lestaurtinib, is a staurosporine derivative. Midostaurin has an FLT3 IC₅₀ of about 10 nM and is described, for example, in Ozaki et al., Anticancer Drug Des. 15:17-28 (2000) and Stone et al., Blood 105:54-60 (2004), each of which is incorporated by reference in its entirety. Midostaurin has the following chemical structure:

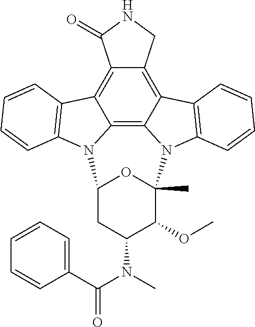 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

Sorafenib is a FLT3 kinase inhibitor that also inhibits several other tyrosine kinase receptors, such as VEGFR and PDGFR. Sorafenib is described, for example, in Auclair et al., Leukemia 21:439-445 (2007) and Metzeldar et al., Blood 113:6567-6571 (2009),), each of which is incorporated by reference in its entirety. Sorafenib has the following chemical structure:

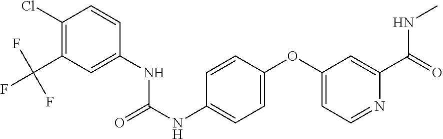 [SMILES: CNC(=O)c1cc(Oc2ccc(NC(=O)Nc3ccc(Cl)c(C(F)(F)F)c3)cc2)ccn1]

KW-2449 has a FLT3 IC₅₀ of about 6 nM and is described, for example, in Pratz et al., Blood 113:3938-3946 (2008) and Shiotsu et al., Blood 114:1607-1617 (2009), each of which is incorporated by reference in its entirety. KW-2449 has the following chemical structure:

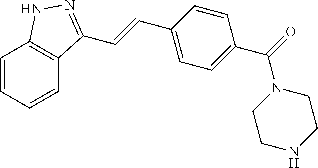 [SMILES: O=C(c1ccc(/C=C/c2n[nH]c3ccccc23)cc1)N1CCNCC1]

AC220 has a FLT3 IC₅₀ of about 4.2 nM and is described, for example, in Chao et al., J Med Chem. 52:7808-7816 (2009) and Zarrinkar et al., Blood 114:2984-2992 (2009), each of which is incorporated by reference in its entirety. AC220 has the following chemical structure:

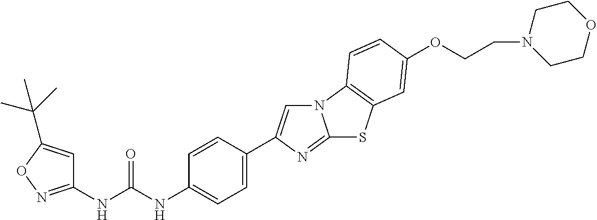 [SMILES: CC(C)(C)c1cc(NC(=O)Nc2ccc(-c3cn4c(n3)sc3cc(OCCN5CCOCC5)ccc34)cc2)no1]

MLN518 has a FLT3 IC₅₀ of about 30 nM and is described, for example, in Griswold et al., Blood. 104:2867-2872 (2004) and DeAngelo et al., Blood 108:3674-3681 (2006), each of which is incorporated by reference in its entirety. MLN518 has the following chemical structure:

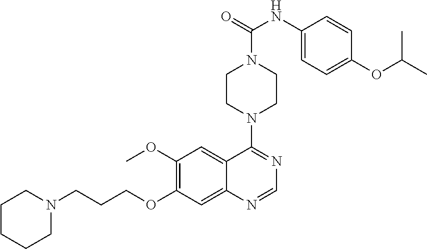 [SMILES: COc1cc2c(N3CCN(C(=O)Nc4ccc(OC(C)C)cc4)CC3)ncnc2cc1OCCCN1CCCCC1]

Sunitnib (SU11248) has a FLT3 IC₅₀ of about 50 nM and is described, for example, in O'Farrell et al., Blood. 101:3597-3605 (2003) and Fiedler et al., Blood 105:986-993 (2005), each of which is incorporated by reference in its entirety. Sunitnib has the following chemical structure:

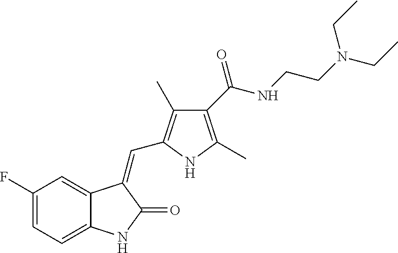 [SMILES: CCN(CC)CCNC(=O)c1c(C)[nH]c(/C=C2\C(=O)Nc3ccc(F)cc32)c1C]

In certain embodiments the FLT3 kinase inhibitors may contain a basic functional group, such as amino or alkylamino, and are, thus, capable of forming pharmaceutically-acceptable salts with pharmaceutically-acceptable acids. These salts can be prepared in situ in the administration vehicle or the dosage form manufacturing process, or by separately reacting a purified agent described herein in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

The pharmaceutically acceptable salts of the subject agents include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. In other cases, the agents described herein may contain one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

### FL and FLT3 Antibodies

In certain embodiments, the present invention relates to the use of antibodies or antigen binding fragments thereof that bind specifically to FL or FLT3. Thus, in some embodiments, the antibodies or antigen binding fragments thereof bind to FL (e.g., human FL, represented by SEQ ID NO: 1) and inhibit FL binding to FLT3. In certain embodiments the antibodies or antigen binding fragments thereof that bind to FLT3 (e.g., human FLT3, represented by SEQ ID NO:2) and inhibit FL binding to FLT3. The antibodies described herein can be polyclonal or monoclonal and can be, for example, murine, chimeric, humanized or fully human.

Polyclonal antibodies can be prepared by immunizing a suitable subject (e.g. a mouse) with a polypeptide immunogen (e.g., a FL protein, a FL polypeptide or a FLT3 extracellular domain polypeptide). For example, FL amino acids 8-15, 81-87 and 116-124 cluster to a single region on the surface of the of the native FL protein and form the site at which FL binds to FLT3 (Graddis et al., The Journal of Biological Chemistry 28:17626-17633 (1998)). Thus, polypeptides comprising one or more of these amino sequences can be used as immunogens to generate anti-FL antibodies that inhibit the binding of FL to FLT3. Similarly, polypeptides comprising one or more if the five immunoglobulin-like domains of the extracellular region of the FLT3 receptor can be used to generate anti-FLT3 antibodies that inhibit the binding of FL to FLT3. See, e.g., U.S. Pat. Pub. No. 2009/0297529, incorporated by reference herein in its entirety. The polypeptide-specific antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to either FLT3 or FL and inhibits the ability of FL to bind to and activate FLT3.

As an alternative to preparing monoclonal antibody-secreting hybridomas, a monoclonal antibody specific for FL or FLT3 can be identified and isolated by screening a recombinant combinatorial immunoglobulin library (e.g., an antibody phage display library or an antibody yeast display library) with the appropriate polypeptide (e.g. a polypeptide having a FL or FLT3 sequence) to thereby isolate immunoglobulin library members that bind the polypeptide. Such library screening techniques can be used to make fully human monoclonal antibodies to FL or FLT3.

Additionally, recombinant antibodies specific for FL or FLT3, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. No. 4,816,567; U.S. Pat. No. 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Fully human monoclonal antibodies specific for FL or FLT3 can also be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N.Y. Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci. 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In certain embodiments, the antibodies of the instant invention are able to bind to FL or FLT3 with a dissociation constant of no greater than 10⁻⁶, 10⁻⁷, 10⁻⁸ or 10⁻⁹ M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis. In some embodiments, the binding of the antibody to FL or FLT3 substantially inhibits the ability of FL to bind to FLT3. As used herein, an antibody substantially inhibits binding of FL to a FLT3 when an excess of polypeptide reduces the quantity of receptor bound to ligand by at least about 20%, 40%, 60% or 80%, 85% or 90% (as measured in an in vitro competitive binding assay).

Exemplary antibodies and antibody fragments that bind to FL and inhibit FL binding to FLT3 are provided, for example in U.S. Pat. No. 7,537,932, which is incorporated by reference in its entirety.

Exemplary antibodies and antibody fragments that bind to FLT3 and inhibit FL binding to FLT3 are provided, for example, in U.S. Pat. Pub. No. 2011/0008355, Piloto et al., Cancer Res. 66:4843-4851 (2006) and Li et al., Blood 103:267-274 (2004), each of which is incorporated by reference in its entirety. Exemplary antibodies include IMC-EB10 and IMC-NC7, which are described, for example, in Zheng et al., Blood 103:267-274 (2004) and Piloto et al., Cancer Res. 65:1514-11522 (2005).

### Soluble FLT3 Receptor Peptides

In certain embodiments, the invention relates to the use of isolated polypeptides comprising the FLT3 extracellular domain or portions of the FLT3 extracellular domain (e.g., comprising one or more of the five immunoglobulin-like (Ig-like) domains, that make up the FLT3 extracellular domain). Such polypeptides are useful, for example, for inhibiting FL binding to FLT3 and for identifying and/or generating antibodies that specifically bind to FLT3 and inhibit FL binding.

In some embodiments, the polypeptide comprises one or more of the five Ig-like domains of the FLT3 extracellular region. In some embodiments, the polypeptide comprises Ig-like domain 4. In some embodiments, the polypeptide comprises Ig-like domains 3-4, 2-4, 1-4, 4-5, 3-5, 2-5 or 1-5. In some embodiments the polypeptide comprises the entire extracellular region of the FLT3 receptor.

In certain embodiments, the polypeptide of the instant invention is able to bind to FL. In some embodiments, the polypeptide binds to FL with a dissociation constant of no greater than 10⁻⁶, 10⁻⁷, 10⁻⁸ or 10⁻⁹. Standard assays to evaluate the binding ability of the polypeptides are known in the art, including for example, ELISAs, Western blots and RIAs and suitable assays are described in the Examples. The binding kinetics (e.g., binding affinity) of the polypeptides also can be assessed by standard assays known in the art, such as by Biacore analysis. In some embodiments, the binding of the polypeptide to FL substantially inhibits the ability of FL to bind to FLT3. As used herein, a polypeptide substantially inhibits binding of FL to a FLT3 when an excess of polypeptide reduces the quantity of receptor bound to ligand by at least about 20%, 40%, 60% or 80%, 85% or 90% (as measured in an in vitro competitive binding assay).

In some embodiments, the polypeptides of the present invention can be isolated from cells or tissue sources by an appropriate purification scheme using standard protein purification techniques. In another embodiment, polypeptides of the present invention are produced by recombinant DNA techniques. Alternatively, polypeptides of the present invention can be chemically synthesized using standard peptide synthesis techniques.

In some embodiments, polypeptides of the present invention comprise an amino acid sequence substantially identical to the sequence of one or more Ig-like domains of the FLT3 extracellular region. Accordingly, in another embodiment, a polypeptide of the present invention is a polypeptide comprises an amino acid sequence at least about 80%, 85%, 90% or 95% identical to a the sequence of an Ig-like domain of the extracellular region of FLT3.

To determine the percent identity of two amino acid sequences, the sequences are aligned for optimal comparison purposes (e.g., gaps can be introduced in one or both of a first and a second amino acid for optimal alignment and non-identical sequences can be disregarded for comparison purposes). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The invention also provides chimeric or fusion proteins. As used herein, a “chimeric protein” or “fusion protein” comprises a polypeptide(s) of the present invention linked to a distinct polypeptide to which it is not linked in nature. For example, the distinct polypeptide can be fused to the N-terminus or C-terminus of the polypeptide either directly, through a peptide bond, or indirectly through a chemical linker. In some embodiments, the polypeptide of the instant invention is linked to an immunoglobulin constant domain.

A chimeric or fusion polypeptide of the present invention can be produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

The polypeptides described herein can be produced in prokaryotic or eukaryotic host cells by expression of polynucleotides encoding a polypeptide(s) of the present invention. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Pharmaceutical Compositions

In certain embodiments, the instant invention relates to pharmaceutical compositions comprising a FLT3 kinase inhibitor and/or an inhibitor of FLT3 ligand binding (e.g., an anti-FL antibody, and anti-FLT3 antibody and/or a soluble peptide comprising one or more Ig-like domains of the FLT3 extracellular region).

In one aspect, the present invention provides pharmaceutically acceptable compositions which comprise a therapeutically-effective amount of one or more of the agents described above, formulated together with one or more pharmaceutically acceptable carriers (additives) and/or diluents. In another aspect the agents of the invention can be administered as such or in admixtures with pharmaceutically acceptable carriers and can also be administered in conjunction with other chemotherapeutic agents. Conjunctive therapy thus includes sequential, simultaneous and separate, or co-administration of the active agent, wherein the therapeutic effects of the first administered has not entirely disappeared when the subsequent agent is administered.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions of this invention suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents of the present invention, which may be used in a suitable hydrated form, and/or the pharmaceutical compositions of the present invention, are formulated into pharmaceutically-acceptable dosage forms by conventional methods known to those of skill in the art.

In certain embodiments, the above-described pharmaceutical compositions comprise one or more of the FLT3 kinase inhibitors and/or FL binding inhibitors and a chemotherapeutic agent, and, optionally a pharmaceutically acceptable carrier. The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); cytarabine, mitoxatrone, antibiotic natural antineoplastics, such as bleomycin, dactinomycin, daunorubicin, doxorubicin, and mitomycin; and vinca alkaloid natural antineoplastics, such as vinblastine and vincristine.

Still further, the following listing of amino acids, peptides, polypeptides, proteins, polysaccharides, and other large molecules may also be used: interleukins 1 through 18, including mutants and analogues; interferons or cytokines, such as interferons α, β, and γ; hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In certain embodiments, the chemotherapeutic agents are mitoxatrone, etoposide and cytarabine. In some embodiments, the chemotherapeutic agent is high dose (greater than 1 g/m² at least twice a day for at least 4 days) cytarabine. In some embodiments, the chemotherapeutic agents are cytarabine, daunorubicin and etoposide.

### Therapeutic Methods

Disclosed herein are novel therapeutic methods of treating leukemia that include administering to a subject, a therapeutically effective amount of a first agent that inhibits the tyrosine kinase activity of FLT3 (a FLT3 kinase inhibitor) and administering to the subject a therapeutically effective amount of a second agent that inhibits the binding of FL to FLT3 (a FL binding inhibitor). A subject in need thereof may include, for example, a subject who has been diagnosed with a leukemia, or a subject who had been previously treated for leukemia, including subjects who have been treated with a FLT3 kinase inhibitor and/or chemotherapy. Examples of chemotherapy are provided herein above.

The methods of the present invention may be used to treat any form of leukemia. In certain embodiments, the leukemia is AML, ALL or CML. In some embodiments the leukemia contains an activating FLT3 mutation, such as an internal tandem duplication in the FLT3 juxtamembrane region or a point mutation in the FLT3 tyrosine kinase domain.

In certain embodiments, the methods of treatment of the present invention include administering an agent that inhibits the binding of FL to FLT3 in conjunction with a second agent (e.g. a FLT3 kinase inhibitor). Conjunctive therapy includes sequential, simultaneous and separate, or co-administration of the active compound in a way that the therapeutic effects of the first compound administered one have not entirely disappeared when the subsequent compound is administered. In some embodiments, a FL binding inhibitor is administered following administration of a FLT3 kinase inhibitor and chemotherapy.

In some embodiments, the instant invention relates to methods of preventing a leukemia relapse in a subject who had previously been treated for leukemia with a first agent that inhibits the tyrosine kinase activity of FLT3, the method including administering to the subject a therapeutically effective amount of a second agent that inhibits the binding of FLT3L to FLT3. In some embodiments the subject had also been previously administered chemotherapy. In certain embodiments, leukemia is AML, ALL or CML. In some embodiments the leukemia contains an activating FLT3 mutation, such as an internal tandem duplication in the FLT3 juxtamembrane region or a point mutation in the FLT3 tyrosine kinase domain.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds of the invention employed in the pharmaceutical composition at levels lower than that required in order to achieve the desired therapeutic effect and gradually increase the dosage until the desired effect is achieved.

### EXEMPLIFICATION

The invention now being generally described will be more readily understood by reference to the following examples which are included merely for purposes of illustration of certain aspects and embodiments of the present invention, and are not intended to limit the invention in any way.

### Materials and Methods

### Clinical Trials

Plasma samples from 5 separate clinical trials of FLT3 inhibitors were used in this study. The Cephalon 204 trial was a randomized trial of lestaurtinib administered in sequence with chemotherapy for AML patients with FLT3 activating mutations in first relapse. Chemotherapy consisted of MEC (mitoxantrone, etoposide, and cytarabine) or HiDAc (high dose cytarabine). A total of 123 plasma samples from 72 patients were used for FLT3 ligand analysis. The MRC AML15 trial was a randomized trial of lestaurtinib administered in sequence with chemotherapy (ADE cytarabine, daunorubicin, etoposide) for newly diagnosed AML patients with FLT3 activating mutations. Patients on AML15 received additional cycles of chemotherapy, each followed by lestaurtinib, for a total of 4 courses. A total of 155 plasma samples from 69 patients were used for analysis of FL levels. The CP00001 trial was a phase I dose escalation trial of AC220 in relapsed or refractory AML patients. Protocol J0509 was an open label dose-escalation study of sorafenib for relapsed or refractory AML patients.

### Patient Samples

Leukemia cell specimens were provided by the Sidney Kimmel Cancer Center at Johns Hopkins Tumor and Cell Procurement Bank. All patients gave informed consent according to the Declaration of Helsinki. The specimens were obtained by Ficoll purifying mononuclear cells from the peripheral blood patients with FLT3/ITD AML. The mononuclear cells were aliquoted and stored frozen in liquid nitrogen in bovine serum with 10% DMSO for repeated use. Prior to each use, aliquots of these blasts were thawed rapidly into warm culture medium, incubated for 12 hours, and then recentrifuged over Ficoll to eliminate cells dying from the freeze-thaw process. Using this method, blasts were obtained that maintained satisfactory viability (as determined by Trypan blue exclusion) and responsiveness to cytotoxic agents in culture over 72 hours.

### Cell Culture

All cell lines and primary blast samples were cultured in culture medium, consisting of RPMI 10% fetal bovine serum (FBS; Millipore, Bedford, Mass.), at 37° C. in 5% CO₂. Ficoll-Hypaque was obtained from GE Healthcare (Piscataway, N.J.). Molm-14 cells were obtained from the DSMZ (Deutsche Sanunlung von Mikroorganismen and Zellkulturen, Germany). The TF/ITD cell line was derived by transfecting the GM-CSF dependent TF-1 cell line with an expression vector containing the FLT3 coding sequence for an ITD mutation from an AML patient, as described. The resultant TF/ITD line was growth factor independent and expresses constitutively phosphorylated FLT3.

Cytotoxicity was assessed using an MTT (Dimethyl-thiazol diphenyl tetrazolium bromide) assay. In selected cases, an Annexin-V binding apoptosis assay was also used to confirm that the cytotoxic effect observed (or lack thereof) was associated with an equivalent degree of apoptosis. MTT (Roche, Indianapolis, Ind.) and Annexin V (Pharmingen, San Diego, Calif.) assays were performed as described previously.

### FLT3 Phosphorylation

Leukemia cells were washed in PBS, then lysed by re-suspending them in lysis buffer (20 mM Tris pH 7.4, 100 mM NaCl, 1% Igepal (Sigma, St. Louis, Mo.), 1 mM EDTA, 2 mM NaV04, plus Complete protease inhibitor (Roche)) for 30 minutes while rocking. The extract was clarified by centrifugation at 14,000 rpm and the supernatant was assayed for protein (Bio-Rad, Richmond, Calif.). A 50 μg aliquot was removed as whole-cell lysate for analysis of STAT5, and the remainder was used for immunoprecipitation with anti-FLT3 antibody. Anti-FLT3 antibody (SI8; Santa Cruz Biotechnology, Santa Cruz, Calif.) was added to the extract for overnight incubation, then protein A sepharose (Upstate Biotechnology, Lake Placid, N.Y.) was added for 2 additional hours. Separate SDS-PAGE (sodium dodecylsulfate polyacrylamide electrophoresis) gels for whole-cell lysate and immunoprecipitates were run in parallel, After transfer to Immobilon membranes (Millipore, Bedford, Mass.), immunoblotting was performed with antiphosphotyrosine antibody (4G10; Upstate Biotechnology, Lake Placid, N.Y.) to detect phosphorylated FLT3, and then stripped and re-probed with anti-FLT3 antibody to measure total FLT3. Proteins were visualized using chemiluminescence (ECL; GE Healthcare, Piscataway, N.J.), exposed on Kodak BioMax XAR Film, developed, and scanned using a BioRad GS800 densitometer. The concentration of drug for which the phosphorylation of FLT3 was inhibited to 50% of its baseline (IC₅₀), or for cytotoxicity, was determined using linear regression analysis of the dose response curves after linear transformation using an exponential model (CalcuSyn software, Biosoft, Inc., Cambridge, U.K.)

### Inhibitors

FLT3 inhibitors were obtained as powder and dissolved in dimethyl sulfoxide (DMSO) at stock concentrations of 10 mM. Stocks were aliquoted into 10 μL volumes and stored at −80° C. and thawed immediately before use. Lestaurtinib was supplied by Cephalon, Inc. (Frazer, Pa.). AC220 was supplied by Ambit Biosciences, Inc. (La Jolla, Calif.). KW-2449 was supplied by Kyowa Kirin, Inc., (Mishima, Japan). Sorafenib and midostaurin were obtained from LC Laboratories, Inc. (Woburn, Mass.). All samples in any given experiment contained identical concentrations of DMSO.

### Plasma Inhibitory Activity Assay

Frozen plasma samples were thawed and clarified by centrifugation at 14,000 rpm for 2 minutes. All assays described herein were performed within 12 months of collection. For each time point, 2×10⁶ TF/ITD were incubated with 1 ml plasma at 37° C. for 1 hour. The cells were washed twice with ice-cold PBS then lysed. After immunoblotting for phosphorylated FLT3 as described above, densitometric analysis was performed on the bands and the PIA for a given plasma sample was calculated by expressing the density of its corresponding band as a percentage of the density of the baseline band (which was arbitrarily set at 100%).

### FL Enzyme-Linked Immunosorbent Assay (ELISA)

FL concentrations in plasma samples were determined using an enzyme-linked immunosorbent assay (ELISA) kit obtained from R&D Systems, Inc. (Minneapolis, Minn., USA).

### Pharmacokinetics

Plasma levels of lestaurtinib in human plasma samples were determined using a validated high-performance liquid chromatography method with fluorescence detection. The method involved liquid-liquid extraction of lestaurtinib from 0.1 mL human plasma into a mixture of ethyl acetate/methylene chloride in a 4:1 (vol/vol) proportion, followed by reverse-phase chromatography on a Hypersil BDS phenyl column.

### Alpha-1 Acid Glycoprotein

Alpha-1 acid glycoprotein was assayed using an immunodiffusion assay kit obtained from Kent Laboratories, Inc. (Bellingham, Wash., USA).

### Reagents

Recombinant human FL was purchased from PeproTech (Rocky Hill, N.J.). Rabbit anti-human FLT3 and anti-STAT5 antibody were obtained from Santa Cruz Biotechnology (Santa Cruz, Calif.). Monoclonal mouse antiphosphotyrosine antibody 4G10 and recombinant protein A-agarose were purchased from Upstate Biotechnology (Lake Placid, N.Y.). Antiphospho-STAT5, anti-phospho-AKT, anti-phospho-MAPK, anti-AKT, and anti-MAPK antibodies were purchased from Cell Signaling Technology (Cell Signaling, Beverly, Mass.). Horseradish peroxidase-conjugated secondary antibody and the enhanced chemiluminescence (ECL) detection system were from Amersham (Arlington Heights, Ill.).

Establishment of FL^{-/-} MEF cells

Embryos were removed from female FL^{-/-} mice (purchased from Taconic, Hudson, N.Y.) on day 13 or 14 of pregnancy. Internal organs and heads were removed. The remainder of the embryos were washed, minced, digested with Trypsin/EDTA, and then resuspended in DMEM containing 10% FBS followed by centrifuging at 1000×g for 5 minutes. The cell pellet was resuspended in DMEM containing 10% FBS and cultured at 37° C. with 5% CO₂. After 3 passages, the cells were transfected with CMV-SV40-large T antigen to immortalize FL^{-/-} MEF cells. The FL^{-/-} MEF cells represent bulk cells after transformation.

### Reverse Transcription Polymerase Chain Reaction (RT-PCR)

Total cellular RNA was extracted from cultured cell lines (RNeasy columns; Qiagen, Valencia, Calif.). RNA (50 ng-100 ng) was reverse transcribed and amplified (One-Step RT-PCR kit; Invitrogen, Carlsbad, Calif.). PCR was performed in the presence of the following primer pairs: 5′-CAAATTCCTCCCTGTTGCTG-3′ and 5′-GGTGGGAGATGTTGGTCTG-3′, specific for murine FL and amplifies a 363-base pair (bp) sequence; 5′-GCTGGTCGTCGACAACGGCTC-3′ and 5′-CAAACATGATCTGGGTCATCTTTTC-3′, specific for β-actin and amplifies a 353-bp sequence. Reverse transcription was performed at 50° C. for 30 minutes. Thirty-five cycles of amplification were then performed at 94° C. for 30 seconds for denaturation, at 55° C. for 30 seconds for annealing, and finally at 72° C. for 1 minute for extension (DNA iCycler; Bio-Rad, Hercules, Calif.). PCR products were resolved on a 1% agarose gel in the presence of ethidium bromide and photographs were taken under UV transillumination.

### DNA Constructs and Retroviral Transduction

The mutant FLT3/ITD sequences were isolated from bone marrow samples from AML patients at our institution by RT-PCR. The ITD fragments were sequenced and used to replace the corresponding wild-type region in full-length FLT3 cDNA. This ITD resulted in an addition of 6 amino acids, RTDFRE, after amino acid No. 596. The FLT3/TKD mutation was obtained by mutagenesis of wtFLT3 to substitute nucleotide G of D835 with T resulting in an Aspartic acid to Tyrosine change (QuickChange Site-Directed Mutagenesis Kit; Stratagene, La Jolla, Calif.). Both types of mutants were then cloned into the pBabePuro retroviral vector and pCIneo expression vector (Promega, Madison, Wis.).

Retrovirus was obtained by harvesting supernatant 48 hours after transfection of 293T cells with pBabePuro constructs. FL^{-/-} MEF cells were cultured with virus in the presence of polybrene (8 μg/mL) for 2 days. Stable transfectants were then selected by limiting dilution in 96-well plates with 2 μg/mL puromycin. Experiments were performed on at least two clones selected from the transfectants.

The fragment containing the secreted form of FL coding sequence was amplified from the pUMVC3-hFLex plasmid by PCR and cloned into the EF1-CMV-GFP lentiviral vector. Viral stocks were generated and transduction of cells was conducted as described above. GFP positive cells were sorted by flow cytometry.

TF1 and BaF3 cells were transfected with pCINeo constructs containing either FLT3/ITD or FLT3/TKD mutation by electroporation (Bio-Rad, Hercules, Calif.). Stable transfectants were then selected by limiting dilution in 96-well plates with 1 mg/mL G418 (Invitrogen, Carlsbad, Calif.).

### Colony-Replating Assay

Bone marrow was harvested from FL^{-/-} mice, lineage depleted (Lineage Cell Depletion kit; Militenyibi Biotec, Auburn Calif.), and transduced with EF1-ITD-UBC-GFP, EF1-TKD-UBC-GFP, or EF1-UBC-GFP lentivirus by 3 rounds of spin-infection on days 1, 2 and 3. The EF1-ITD-UBC-GFP vector was constructed by inserting full-length FLT3/ITD in EF1-UBC-GFP lentiviral vector. The EF1-TKD-UBC-GFP was constructed by inserting wtFLT3 into the EF1-UBC-GFP followed by mutagenesis of wtFLT3 to substitute nucleotide G of D835 with T resulting in an Aspartic acid to Tyrosine change (QuickChange Site-Directed Mutagenesis Kit; Stratagene, La Jolla, Calif.). GFP positive cells were then sorted by FACS. 10³ cells were plated into triplicate 35 mm Petri dishes (Nunc) in 1 ml of Methocult M3434 (StemCell Technologies Inc., Vancouver) containing 50 ng/mL rm SCF, 10 ng/mL rm IL-3, 10 ng/mL rh IL-6, and 3 units/mL rh Erythropoietin. Cultures were incubated at 37° C. in a humidified atmosphere of 5% CO₂, and colonies consisting of >35 cells were scored after 9-11 days. For the colony-replating assay all cells from the previous culture were collected and re-plated at 1×10⁴ cells/plate in fresh methylcellulose medium. All cells were replated three times.

Generation of Mice with FL^{-/-}ITD^{+/+} and FL^{+/+}/ITD^{+/+} Genotype

FL^{-/-}FLT3 wt/wt C57/B16 mice were crossed with FL^{+/+}FLT3 wt/ITD C57/B16 knock-in mice previously generated in the lab (Li et al., 2008). Heterozygotes with the genotype of FL^{+/+}FLT3 wt/ITD were then crossed to generate the desired FL^{-/-}ITD^{+/+} and FL^{+/+}/ITD^{+/+} mice. Genotypes were analyzed by PCR with the following primers: ITD, 5′-CTCTCGGGA ACTCCCACTTA-3′,5′-TGCAGATGATCCAGGTGACT-3; FL, 5′-GCCCAA ATGTGCGTATACCT-3′,5′-CCCAGCACAGTATGGGAACT-3′; NeoFL, 5′-ACACTTCGAAGCTGGAAAGC-3′,5′-GGGGAACTTCCTGACTAGGG-3′. Survival of FL^{-/-}ITD^{+/+} and FL^{+/+}/ITD^{+/+} mice were followed up to 525 days. White blood cell (WBC) differential counting was performed by collecting 50 μL peripheral blood and subjecting it to analysis using a Hemavet950 Hematology system (Drew Scientific, Oxford, Conn.). Murine internal organs were dissected from moribund mice and subjected to gross examination. Bone marrow was collected and subjected to histological and flow cytometry analysis. All animal procedures were conducted in conformity with institutional guidelines.

### Apoptosis and Survival Assay

Evaluation of apoptosis was performed by annexin V/7-AAD staining according to the recommendations of the manufacturer (Becton Dickinson, San Jose, Calif.). After serum starvation, 5×10⁵ cells were washed with cold PBS and re-suspended in 100 μL of binding buffer. Following incubation with 5 μL of annexin V-PE and 5 μL of 7-AAD in the dark for 15 minutes at room temperature, cells were analyzed by flow cytometry (FACSort, Becton Dickinson) with Cell Quest software (Becton Dickinson). Compensation was performed with samples stained with either annexin V-PE or 7-AAD alone.

2×10⁴ cells in 100 μl serum-free medium were plated into triplicate wells of 96-well plates. Survival was assessed by MTT assay (Roche, Indianapolis, Ind.) and viable cell number counting by trypan blue (GIBCO, Rockville, Md.) exclusion according to the manufacturer's instructions.

### Example 1

### FLT3 Inhibition is More Effective in Newly Diagnosed Patients than Relapsed Patients

Using a plasma inhibitory activity (PIA) assay for FLT3, in vivo FLT3 inhibition in AML patients treated with chemotherapy followed by lestaurtinib was examined, comparing newly-diagnosed AML patients (enrolled on the MRC AML15 trial) with relapsed patients enrolled in the Cephalon 204 trial. Both groups of patients had been treated with a 3 drug regimen followed by 80 mg twice daily of lestaurtinib. Plasma samples collected at trough time points on Day 15 following the beginning of chemotherapy were used to measure both PIA data and lestaurtinib drug levels (78 from the 204 trial, 18 from the AMLI5 trial). FLT3 inhibition by lestaurtinib was distinctly less effective in the relapsed patients (204 trial) compared with the newly-diagnosed patients (AML15 trial). Preliminary data from both trials indicated that FLT3 inhibition to a level of less than 15% of baseline at the day 15 time point was strongly correlated with remission in both groups of patients. As summarized in FIG. 3, the mean level of FLT3 activity on Day 15 for patients in the AML15 trial was significantly (p=0.0001) less than for those in the 204 trial. Both groups of patients were receiving the same dose of lestaurtinib, and samples were collected 12 hours after dosing on or around Day 15. In vitro and early phase clinical studies of lestaurtinib and other FLT3 inhibitors indicated that FLT3 autophosphorylation generally must be suppressed to below 15% of baseline in order to induce a cytotoxic effect in AML cells. Out of 78 samples from the 204 trial analyzed, only 48 (62%) achieved this target level of inhibition, whereas all 18 of the AML15 samples analyzed were below 15% of baseline FLT3 activity. Results of previous monotherapy trials of lestaurtinib indicated that trough levels of 5 μM in vivo would be sufficient to induce FLT3 inhibition to 15% of baseline. However, despite a mean plasma trough level of 12.3 μM (FIG. 3) inhibition in the 204 trial patients was significantly less effective than for those in the AML15 trial.

### Example 2

### Plasma FLT3 Ligand Levels are Elevated Following Chemotherapy

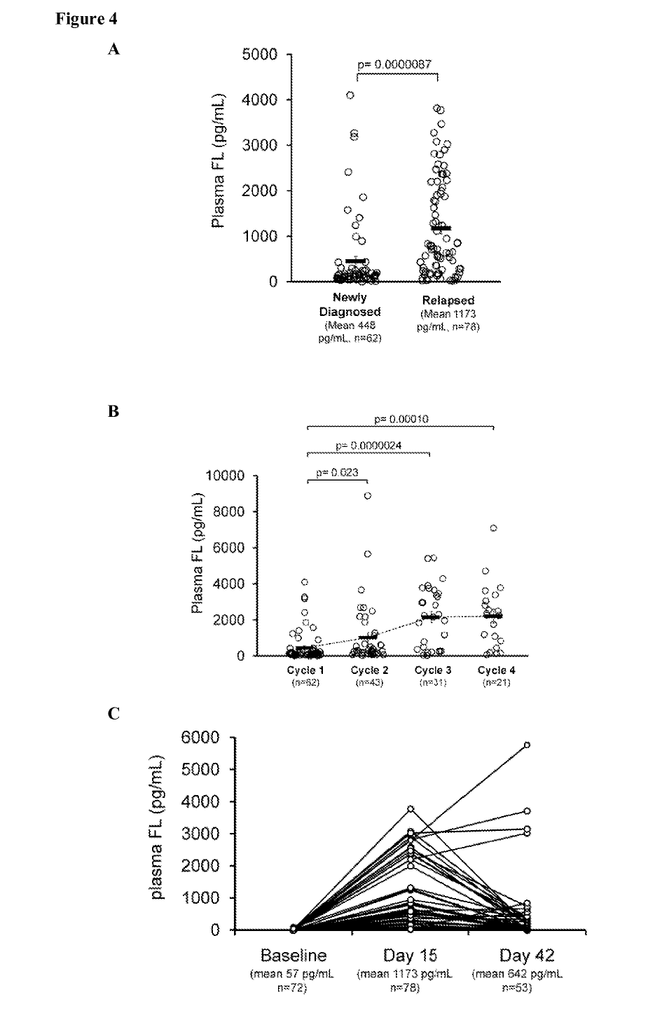

FL levels were examined in the plasma samples obtained during the clinical trials described above. To derive an estimate of FL levels in newly diagnosed AML patients, plasma from 7 AML patients was obtained at diagnosis. The FL levels for these 7 patients ranged from 2 to 6 pg/mL (mean 3 pg/mL). For AML patients at first relapse, 72 baseline plasma samples from the Cephalon 204 trial were tested. In these patients there was considerably more variation in FL levels, ranging from 2 pg/mL to as high as 2953 pg/mL (mean 57 pg/mL). Shown in FIG. 4A are the individual and mean FL levels for Day 15 of course 1 of induction therapy for the newly diagnosed AML15 patients compared with Day 15 of salvage chemotherapy for the relapsed Cephalon 204 trial patients. While there is wide individual variation in FL levels in both trials, as a group, the relapsed patients have significantly higher levels. Plasma samples available from subsequent courses of chemotherapy from the AML15 trial (all on Day 15 of each course) were also tested, and mean FL levels rose higher with each subsequent course (FIG. 4B). The FL level on Day 15 of the second course of chemotherapy for the AML15 patients was comparable in magnitude to that seen in the relapse patients on Day 15 of their salvage therapy. From the Cephalon 204 trial, plasma samples collected on or around Day 42 from individual patients during recovery from a single course of chemotherapy were tested. Results from these samples (FIG. 4C) indicated that FL levels remain elevated for weeks after initiation of therapy.

To determine the effect of FLT3 inhibition in vivo on FL plasma levels (alone, in the absence of chemotherapy), plasma samples from patients treated with two other FLT3 inhibitors, sorafenib or AC220 were tested. Samples were obtained from 16 patients with in vivo FLT3 inhibition to less than 15% of baseline for at least 2 continuous weeks. The levels of FL in these patients ranged from 4 to 155 pg/mL (mean 32 pg/mL), essentially the same as those seen in untreated patients, and roughly 2 orders of magnitude less than the levels seen in patients following chemotherapy.

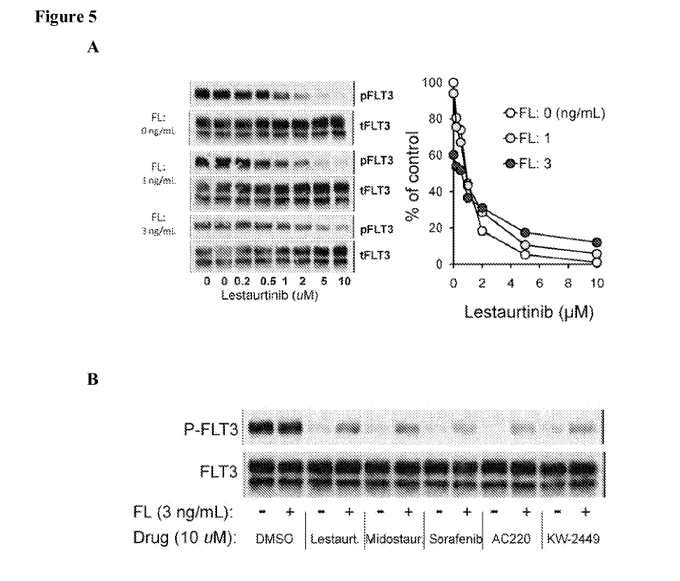

The effects of FL on the efficacy of FLT3 inhibitors were characterized in vitro. Molm14 cells, which harbor a FLT3/ITD mutation, were incubated in increasing concentrations of lestaurtinib in the presence of 0, 1, or 3 ng/mL of exogenous FL. These FL concentrations were chosen because they were similar to those measured on Day 15 in the Cephalon 204 trial patients. As shown in FIG. 5A, FL induced an upward shift in the dose response curve for inhibition of FLT3 autophosphorylation by lestaurtinib both in medium and plasma. There was a down-regulation of phosphorylated and total FLT3 in response to the addition of FL (presumably due to receptor internalization), but at higher doses of lestaurtinib FLT3 autophosphorylation was still evident compared to the same experiment performed in the absence of FL. At concentrations of 10 μM lestaurtinib in plasma, which is a level frequently observed in the trial patients, 3 ng/mL FL resulted in significant residual FLT3 autophosphorylation (FIG. 5A).

Similar dose-response experiments were performed in medium and plasma for other FLT3 inhibitors (sorafenib, AC220, PKC412, and KW-2449). In all cases, the addition of FL resulted in loss of complete inhibition by these agents at the highest doses used, and an upward shift in the dose response curves. Because of receptor internalization in the presence of FL (see FIG. 5A), the dose response curves were influenced by two factors (FL and the inhibitors) and therefore did not have first order kinetics. FIG. 5B illustrates the effect that the addition of 3 ng/mL FL had on the inhibitory efficacy of all of these drugs in plasma.

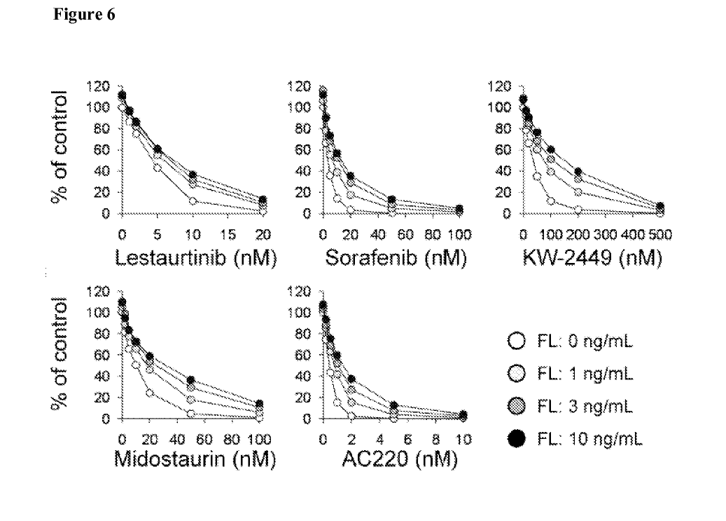

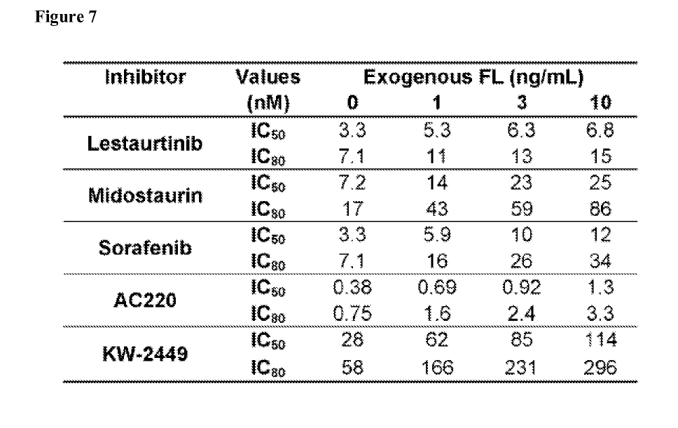

Since the intent of inhibiting FLT3 autophosphorylation in AML is to induce a cytotoxic response, it was determined whether the cytotoxic effects of these FLT3 inhibitors were also mitigated by the addition of FL. Molm14 cells were incubated for 48 hours with increasing concentrations of FLT3 inhibitors in the presence increasing concentrations of FL, and then assayed for cytotoxic effect (FIG. 6). For all drugs analyzed, FL increased the IC₅₀ and IC₈₀ values for cytotoxicity. The results are summarized in FIG. 7.

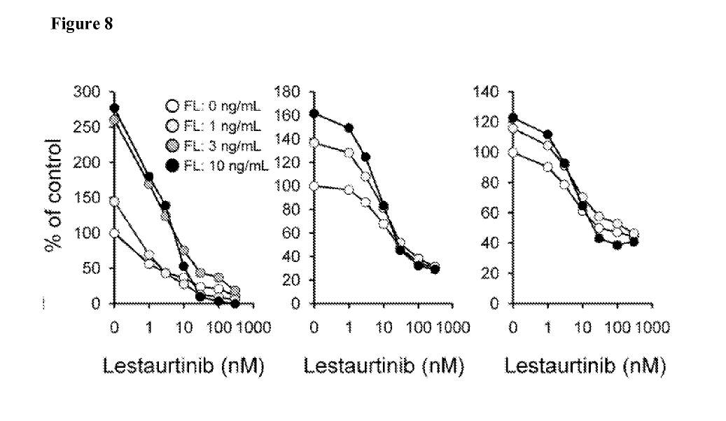

In three primary samples from AML patients with FLT3/ITD mutations (FIG. 8), a similar pattern of response to FL and FLT3 inhibition was observed. For all 3 samples, the addition of exogenous FL increased the metabolic activity of the sample in culture medium, and resulted in a relative upward shift in the cytotoxicity dose response curve.

### Example 3

### Increased FL Levels are Associated with Decreased FLT3 Inhibition

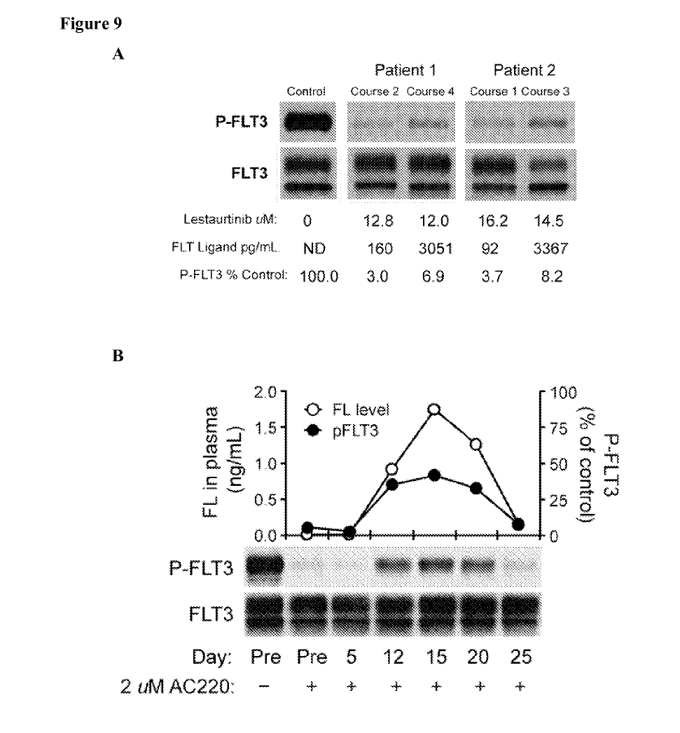

Because FL levels continue to rise with successive courses of chemotherapy, it was investigated whether in vivo FLT3 inhibition by lestaurtinib would be less effective after the first chemotherapy course. The mean PIA value from course 1 of the AML15 trial (18 samples total analyzed) was 3% of baseline, while in courses 2 through 4 (18 samples total) the value was 6.8% (p=0.04). In order to better establish an association between elevated FL levels and impairment of FLT3 inhibition, the data set for cases in which individual patients had similar plasma concentrations of lestaurtinib at two different time points during treatment, but different levels of FL were examined. Shown in FIG. 9A are two such cases. In both examples, the FL concentration was above 3 ng/mL, and in both cases the intensity of FLT3 autophosphorylation was more than 2-fold higher.

In order to better characterize the effects of chemotherapy-induced FL increases on FLT3 inhibition within a single patient, plasma was collected from a newly-diagnosed AML patient (without a FLT3 mutation) at diagnosis, and then every 5 days following induction chemotherapy. The chemotherapy regimen consisted of cytarabine 667 mg/meter squared/day by continuous infusion on Days 1-3, daunorubicin 45 mg/meter squared on Days 1-3, and etoposide 400 mg/meter squared/day on days 8-10. This was a more dose-intense regimen than either of the ones used in the lestaurtinib trials. FL levels were measured in the plasma from these time points (FIG. 9B), and then, in a parallel experiment, the FLT3 inhibitor AC220 was added, to a concentration of 2 μM to each plasma time point sample. The AC220-containing plasma was then used to incubate Molm14 cells, and phosphorylated FLT3 was then evaluated by immunoblot. As shown in FIG. 9B, FL levels rose following chemotherapy, reach a peak on Day 15, then fell back near baseline by Day 25. In parallel with the FL curve, the inhibitory activity of AC220 was significantly blunted between days 12 and 20.

### Example 4

### FL Addition Leads to Increased Phosphorylation of FLT3 in FL^{-/-} MEF Cells Expressing wtFLT3, FLT3/ITD, and FLT3/TKD

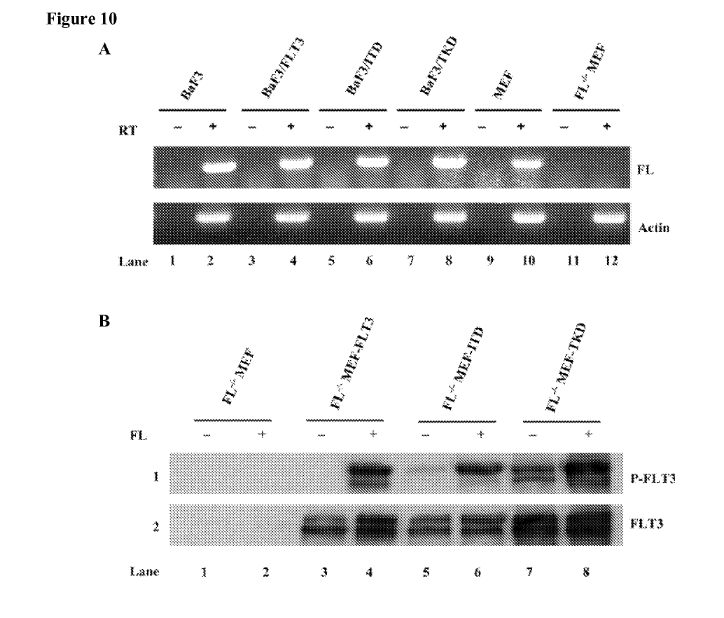

To eliminate the complicating factor of autocrine, paracrine, and intracrine FL expression on mutant FLT3 activation, FL^{-/-} MEF cells were established from FL^{-/-} mice. The FL deficiency in the established FL^{-/-} MEF bulk cells was confirmed by RT-PCR analysis (FIG. 10A). Absence of murine wtFLT3 expression was confirmed by Western blotting. The FL^{-/-} MEF cells were then transduced by a lentiviral vector containing the wt, ITD or TKD forms of FLT3 and clones were selected by limited dilution. Stable expression was confirmed by Western blotting (FIG. 10B). Cells were then cultured in serum-free medium (to eliminate the effect of FL contained in serum) for 16 hours before stimulation with and without FL. The addition of FL induced more than a 4-fold increase in FLT3 phosphorylation in FL^{-/-} MEF-FLT3, FL^{-/-} MEF-ITD, and FL^{-/-} MEF-TKD cells (quantitated by GS-800 Calibrated Densitometer (Bio-Rad) and Quantity One software (Bio-Rad)) (FIG. 10B, lanes 4, 6, and 8 versus lanes 3, 5, and 7). There was no phosphorylation of FLT3 in FL^{-/-} MEF-FLT3 and the phosphorylation of the FLT3 mutants in FL^{-/-} MEF cells was significantly less in the absence of FL stimulation (FIG. 10B, lanes 3, 5, and 7). Similar results were obtained from experiments using both bulk cells (FIG. 10B) and additional clones (data not shown). These data indicate that the addition of FL leads to increased activation of FLT3/ITD and FLT3/TKD receptors.

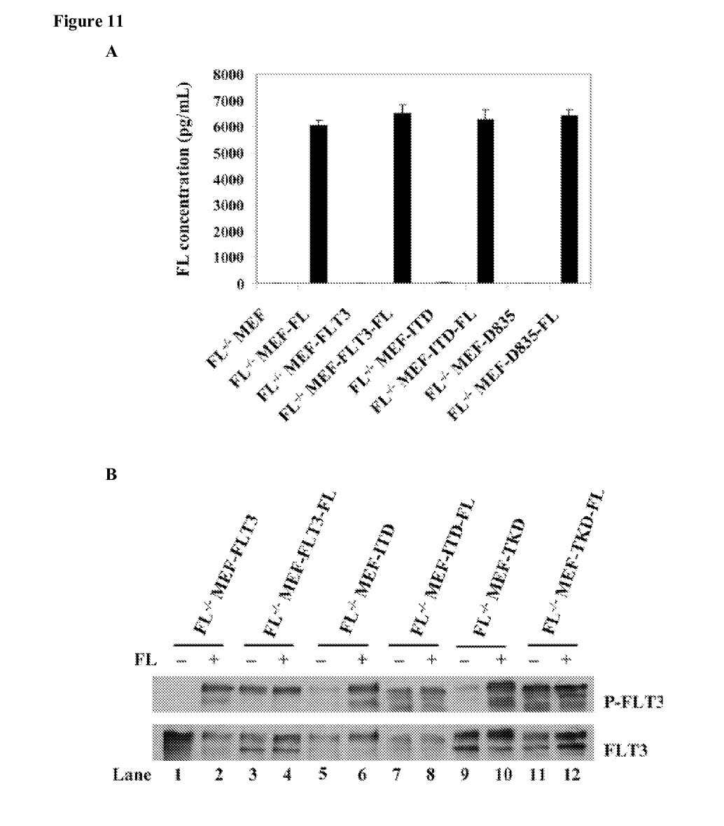

The effect of endogenous FL expression on the activation of FLT3/ITD and FLT3/TKD mutant receptors were next examined. FL was introduced into different clones of FL^{-/-} MEF-FLT3, FL^{-/-} MEF-ITD, and FL^{-/-} MEF-TKD cells by lentiviral transduction to simulate FL expression by leukemia blasts and cell lines. The expression of FL was assessed by the detection of the secreted form of FL in the cell culture supernatant. The level of secreted FL was comparable in FL^{-/-} MEF-FLT3, FL^{-/-} MEF-ITD, and FL^{-/-} MEF-TKD cells expressing FL (FIG. 11A). These cells were cultured in serum-free medium followed by stimulation with and without FL. Endogenous expression of FL resulted in a more than 3-fold increase in FLT3 phosphorylation in FL^{-/-} MEF-FLT3, FL^{-/-} MEF-ITD, and FL^{-/-} MEF-TKD cells in the absence of exogenous FL (FIG. 11B, lanes 3, 7, and 11 vs. 1, 5, and 9). The addition of exogenous FL (100 ng/mL) did not further augment FLT3 phosphorylation in FL^{-/-} MEF-FLT3, FL^{-/-} MEF-ITD, or FL^{-/-} MEF-TKD cells expressing endogenous FL (FIG. 11B, lanes 4, 8, and 12 vs. lanes 3, 7, and 11). These results indicate that endogenous FL expression strongly contributes to the observed activation of mutant FLT3 receptors.

### Example 5

### Exogenous FL Augments the Phosphorylation of FLT3 and Enhances Survival and Resistance to Apoptosis in Cell Lines Expressing FLT3 Mutants

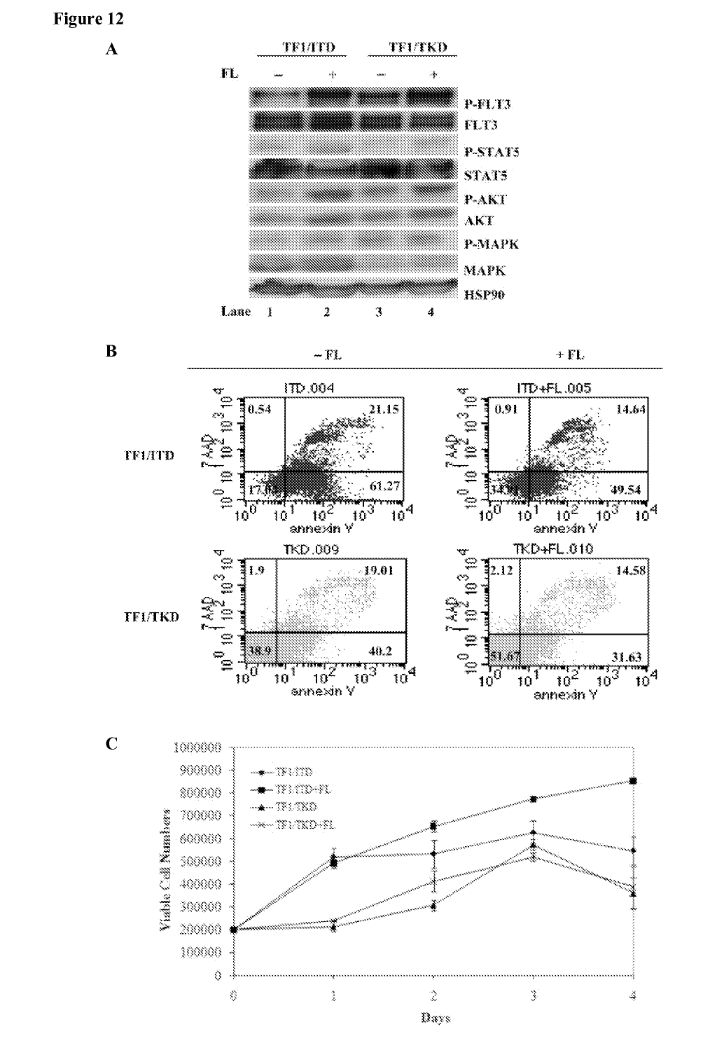

The possibility of further activation of FLT3 mutant receptors by exogenous FL was also tested in cell lines in which FL autocrine, paracrine, or intracrine stimulation of FLT3 receptor might exist. TF1 cells that do not express FLT3 receptor were transfected with constructs containing mutant forms of FLT3. TF1 cells expressing FLT3 mutant receptors were starved for 16 hours in serum-free medium and washed once with PBS to deplete soluble FL (either produced by the cells or contained in the serum). These cells were then stimulated with exogenous FL for 15 minutes. More than 2-fold enhancement of FLT3 phosphorylation was detected in both TF1/ITD and TF1/TKD cell lines (FIG. 12A). Slightly increased STAT5 phosphorylation (1.8-fold increase in both TF1/ITD and TF1/TKD), AKT phosphorylation, and AKT protein expression was also observed after FL stimulation (FIG. 12A). To assess the functional effect of exogenous FL, TF1/ITD and TF1/TKD cells were cultured in serum-free medium in the absence and presence of FL for 48 hours. Apoptosis was assessed by Annexin V and 7AAD staining followed by FACS analysis. The presence of FL decreased the apoptotic population from 61.27% to 49.54% and from 40.2% to 31.63% in TF1/ITD and TF1/TKD cells, respectively (FIG. 12B). There was no change of cell cycle status in response to FL in these cells (data not shown). Survival assays performed by counting viable cell numbers revealed that exogenous FL increased the survival of TF1/ITD cells cultured in serum free medium (FIG. 12C, P<0.05). However, no significant difference was observed in TF1/TKD cells in the presence and absence of FL (FIG. 12C). This data suggests that the presence of FL augments the activation of FLT3/ITD mutant receptors and can contribute to resistance to apoptosis.

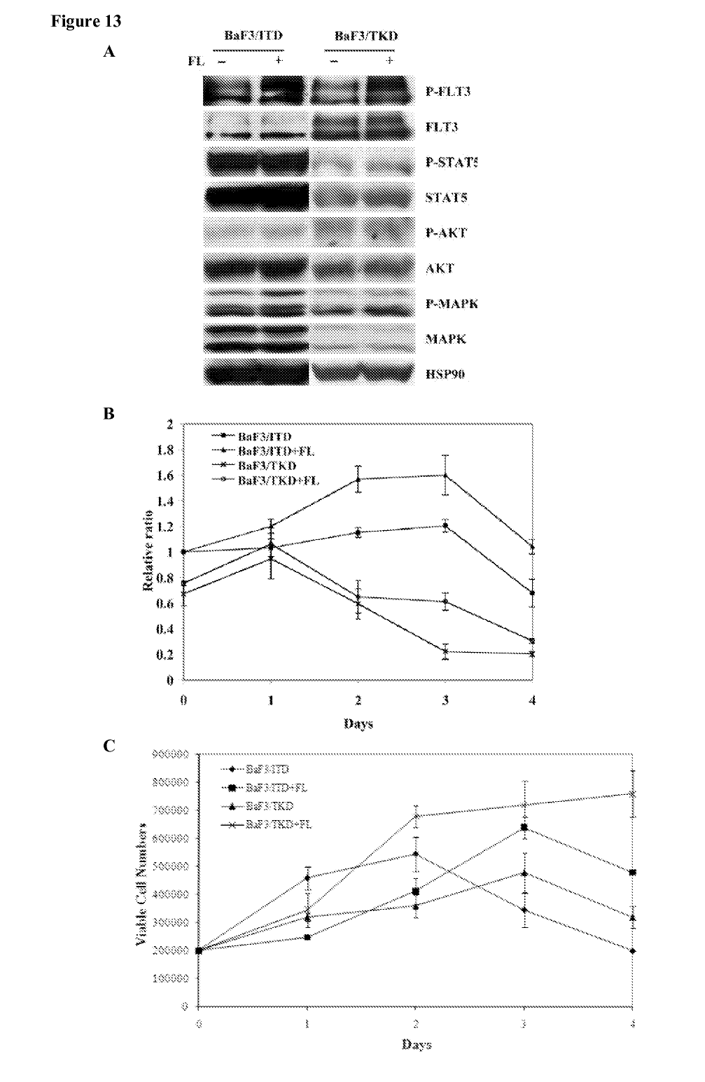

BaF3 is another cell line lacking expression of endogenous FLT3. BaF3 cells transfected with mutant forms of FLT3 were starved in serum-free medium for 16 hours and washed with PBS before FL stimulation. The addition of FL increased FLT3 phosphorylation in both BaF3/ITD and BaF3/TKD cells. An increase of MAPK phosphorylation was also observed (FIG. 13A). In both BaF3/ITD and BaF3/TKD cells, exogenous FL also led to slightly increased phosphorylation of AKT (1.3 and 2.2-fold increase in BaF3/ITD and BaF3/TKD, respectively, FIG. 13A). Changes of other FLT3 downstream signals were not observed. An MTT survival assay was performed in BaF3/ITD and BaF3/TKD cells cultured in serum-free medium in the presence and absence of FL for 4 days. FL addition resulted in increased survival of BaF3/ITD and BaF3/TKD cells (FIG. 4B, P<0.05). The effect was also confirmed by counting viable cell numbers by trypan blue exclusion (FIG. 13C, P<0.05). These findings indicate that FL stimulation prolongs the survival of cells expressing FLT3 mutants.

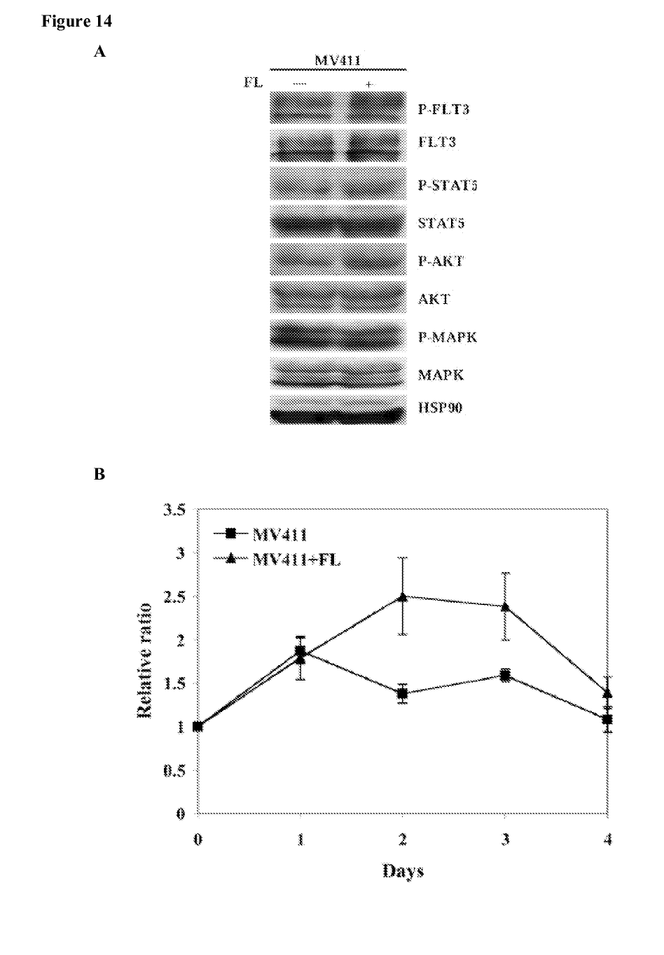

The MV411 cell line is a human leukemia-derived cell line expressing a FLT3/ITD mutation and has lost the wild-type allele. In agreement with the results seen in TF1 and BaF3 transfectants, exogenous FL resulted in increased phosphorylation of FLT3 in MV411 cells (FIG. 14A). Slightly enhanced phosphorylation of FLT3 downstream signaling, including STAT5 and AKT, was also detected after FL stimulation (FIG. 14A). MV411 cells express endogenous FL that might diminish the degree of the effect of exogenous FL on FLT3/ITD mutant receptor. To evaluate the effect of FL on the survival of MV411 cells, MV411 cells were cultured in serum-free medium in the presence and absence of FL for 4 days. FL addition led to prolonged survival of MV411 cells (FIG. 14B, P<0.05).

### Example 6

### FL Addition Results in Increased Phosphorylation of Mutant FLT3 Receptors and Prolongs Survival of Primary AML Blasts Expressing Homozygous FLT3/ITD Mutations

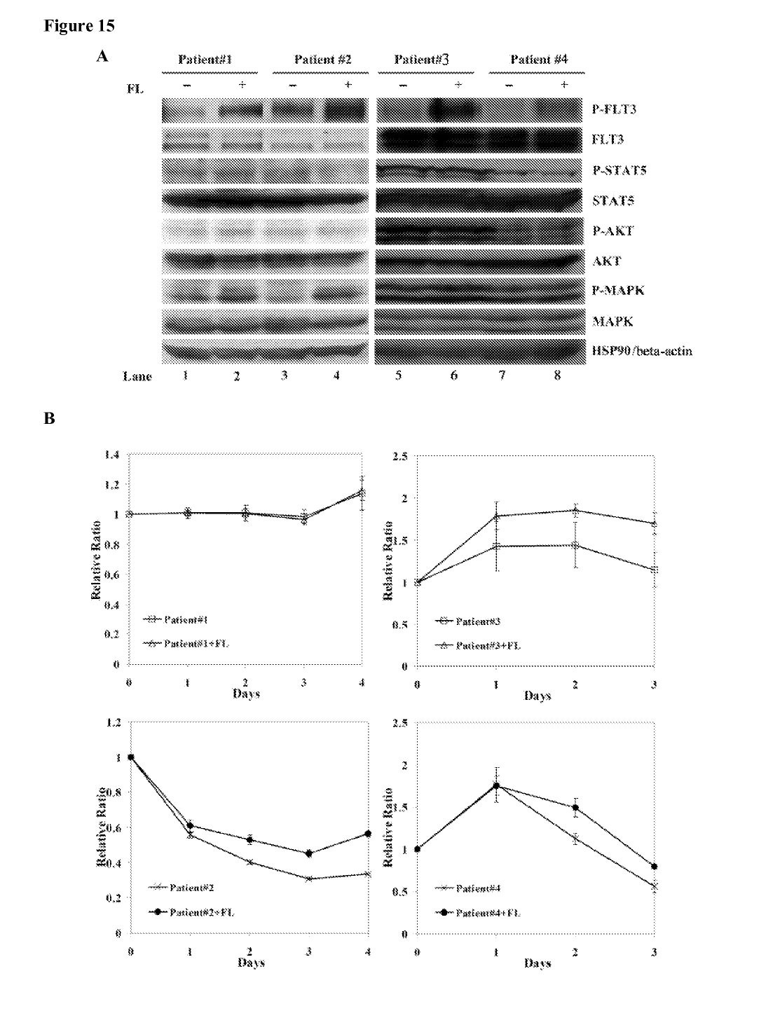

To study whether further activation of mutant FLT3 receptors in response to FL also occurs in primary cells, experiments were carried on primary AML blasts expressing homozygous FLT3/ITD mutations (i.e., no detectable wild type FLT3 by PCR). The blasts were stimulated with 100 ng/mL FL. Consistent with what we observed in the FLT3 mutant cell lines, exogenous FL caused enhanced FLT3 phosphorylation (FIG. 15A). Increased MAPK phosphorylation was also detected in two out of four samples (patient 1 and 2). The survival of AML blasts was assessed by culturing them in complete medium with and without FL for 4 days. The presence of FL increased the survival of AML blasts derived from three of four patient samples (FIG. 15B, P<0.05 in patient #2, 3 and 4).

### Example 7

### FL Stimulation Increases the Clonogenic Potential of FL^{-/-} Primary Hematopoietic Cells Expressing FLT3/ITD or FLT3/TKD Mutations

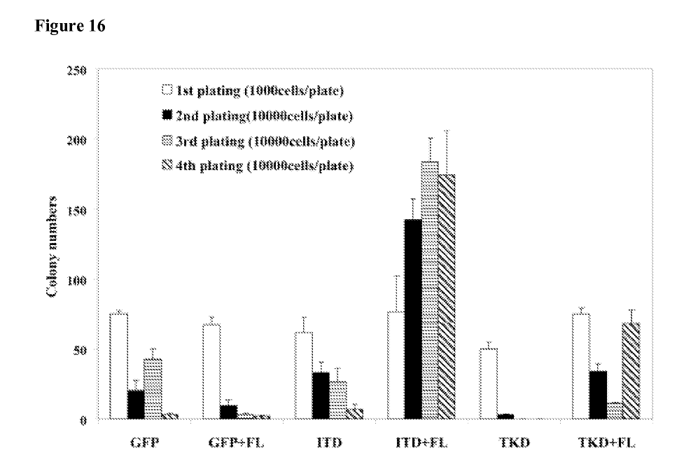

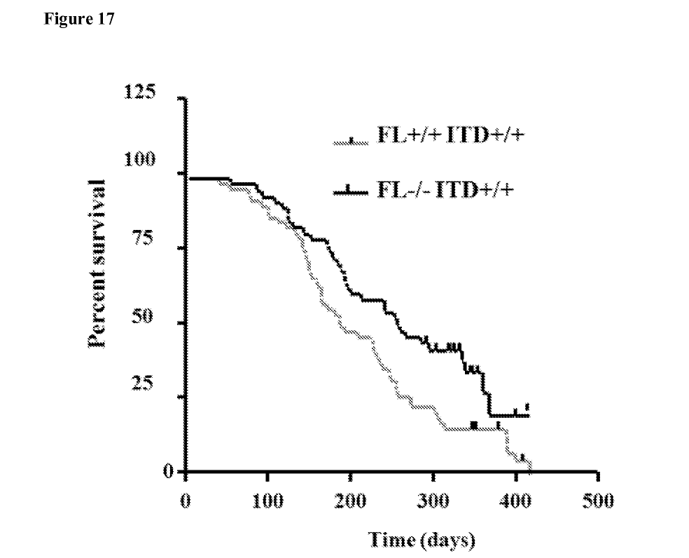

To evaluate the effect of FL stimulation on primary hematopoietic progenitor cells expressing FLT3 mutants, lineage negative progenitors were harvested from FL^{-/-} mice and transduced with FLT3/ITD-GFP, FLT3/TKD-GFP, or GFP control lentivirus. GFP-positive cells were selected by FACS, cultured in methylcellulose with and without FL, and the colony numbers were counted after 9 days. The number of first-generation colonies did not differ significantly between ITD, TKD and control vector transduced bone marrow (BM) cells when cultured in the absence of FL stimulation (FIG. 16). Exogenous FL slightly increased the number of first-generation colonies derived from BM cells transduced with FLT3 mutants (FIG. 16).

After colonies were counted, all of the cells in each culture dish were harvested from the methylcellulose, washed and re-plated in fresh methylcellulose medium to examine their potential to form colonies upon secondary plating; this process was repeated for two more passages to determine the frequency of tertiary and quaternary colonies. Control FL^{-/-} BM cells transduced with GFP control vector alone cultured in the absence and presence of FL exhausted virtually all of their proliferative potential by the fourth passage (FIG. 16). Upon serial re-plating, the number of colonies from FLT3/ITD and FLT3/TKD transduced FL^{-/-} BM cells cultured in the absence of FL also decreased with each passage (FIG. 16). In contrast, BM cells from FL^{-/-} mice transduced with FLT3 mutants and cultured in the presence of FL retained their re-plating activity and continued to produce colonies despite repeated passages (FIG. 16, P<0.05). Besides the increase in colony numbers, there was also a significant increase in colony size from FL^{-/-} BM cells expressing FLT3 mutants stimulated with FL (data not shown). These results indicate that FL stimulation is able to confer a proliferative advantage and immortalized potential on hematopoietic progenitors expressing FLT3 mutants.

### Example 8

### Endogenous FL Shortens the Survival of FL^{+/+}ITD^{+/+} Mice in Comparison with FL^{-/-}ITD^{+/+} Mice

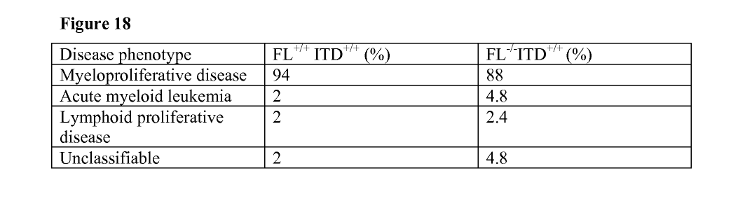

FLT3/ITD knock-in mice develop myeloproliferative disease (MPD) which eventually progressed to mortality by 6 to 20 months. To determine whether or not expression of FL would have any functional effect on survival, the FL^{-/-}FLT3 wt/wt mice were crossed with FL^{+/+}FLT3 wt/ITD mice followed by cross of the second generation double heterozygotes to generate FL^{+/+}ITD^{+/+}, and FL^{-/-}ITD^{+/+} mice. 51 FL^{+/+}ITD^{+/+} mice and 32 FL^{-/-}ITD^{+/+} mice were followed for the development of fatal MPD and the median survival of the two groups were 187 and 256 days, respectively (P<0.05, FIG. 17). Thus, the presence of endogenous FL significantly decreased the survival of homozygous ITD mice (FL^{+/+}ITD^{+/+} mice vs. FL^{-/-}ITD^{+/+} mice, P<0.05). The majority of the mice in both groups developed splenomegaly and leukocytosis (data not shown). However, the spleen weight and white blood cell count did not differ significantly between the two groups (P<0.05). By determination of the phenotype by FACS analysis of the bone marrow, in conjunction with blood counts, spleen weight and bone marrow morphology, 88% of the FL^{-/-}ITD^{+/+} mice and 94% of the FL^{+/+}ITD^{+/+} mice developed myeloproliferative disease (FIG. 18). The in vivo data indicate a functional role for FL in accelerating the progress of fatal MPD.

### EQUIVALENTS

While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entirety as if each individual publication or patent was specifically and individually indicated to be incorporated by reference. In case of conflict, the present application, including any definitions herein, will control.

1. A method of treating leukemia in a subject comprising administering to the subject a therapeutically effective amount of a first agent that inhibits the tyrosine kinase activity of FLT3 and administering to the subject a therapeutically effective amount of a second agent that inhibits the binding of FLT3L to FLT3.

2. The method of claim 1, wherein the first agent is a small molecule.

3. The method of claim 2, wherein the first agent is lestaurtinib, midostaurin, sorafenib, KW-2449, or AC220.

4. The method of claim 1, wherein the second agent is an antibody or antibody fragment that specifically binds to FLT3L.

5-7. (canceled)

8. The method of claim 1, wherein the second agent is a polypeptide comprising a portion of the FLT3 extracellular domain.

9. The method of claim 8, wherein the polypeptide further comprises an immunoglobulin constant domain or portion thereof.

10. The method of claim 1, further comprising administering a chemotherapy to the subject.

11. The method of claim 10, wherein the chemotherapy comprises:
administering mitoxatrone, etoposide and cytarabine to the subject;
administering high dose cytarabine to the subject; or
administering cytarabine, daunorubicin and etoposide to the subject.

12. The method of claim 1, wherein leukemia cells in the subject comprise a FLT3 activating mutation.

13. The method of claim 12, wherein the FLT3 activating mutation is an internal tandem duplication in the FLT3 juxtamembrane region or a point mutation in the FLT3 tyrosine kinase domain.

14. The method of claim 1, wherein the leukemia is AML, ALL or CML.

15. The method of claim 1, wherein the administration of the second agent occurs after the administration of the first agent.

16. A method of preventing a leukemia relapse in a subject who had previously been treated for leukemia with a first agent that inhibits the tyrosine kinase activity of FLT3, the method comprising administering to the subject a therapeutically effective amount of a second agent that inhibits the binding of FLT3L to FLT3.

17. The method of claim 16, wherein the first agent is a small molecule.

18. The method of claim 17, wherein the first agent is lestaurtinib, midostaurin, sorafenib, KW-2449, or AC220.

19. The method of claim 16, wherein the second agent is an antibody or antibody fragment that specifically binds to FLT3L.

20. (canceled)

21. The method of claim 16, wherein the second agent is an antibody or antibody fragment that specifically binds to FLT3.

22-24. (canceled)

25. The method of claim 16, wherein the previous leukemia treatment further comprised administration of a chemotherapy to the subject.

26. (canceled)

27. The method of claim 16, wherein the leukemia cells of the subject comprise a FLT3 activating mutation.

28-29. (canceled)

30. A kit for treating leukemia in a subject comprising a therapeutically effective amount of a first agent that inhibits the tyrosine kinase activity of FLT3 and a therapeutically effective amount of a second agent that inhibits the binding of FLT3L to FLT3.

31-40. (canceled)
